(19) United States
(12) Patent Application Publication
(10) Pub. No.: US 2016/0002620 A1
(43) Pub. Date: Jan. 7, 2016

(54) METHOD FOR DIGITAL TRANSDUCTION OF DNA IN LIVING CELLS

(71) Applicant: Luc Montagnier, New York, NY (US)

(72) Inventor: Luc Montagnier, New York, NY (US)

(21) Appl. No.: 14/792,039

(22) Filed: Jul. 6, 2015

Related U.S. Application Data
(60) Provisional application No. 62/020,796, filed on Jul. 3, 2014.
(60) Provisional application No. 62/039,046, filed on Aug. 19, 2014.

(51) Int. Cl. C12N 13/00

(52) U.S. Cl. CPC C12N 13/00

(57) ABSTRACT

A system and method for inducing cytotoxicity, comprising a receiver configured to receive an electromagnetic signal from a container, using a receiver configured to capture electromagnetic emissions from the container over a frequency range of at least 100 Hz to 10,000 Hz; an amplifier configured to amplify the received electromagnetic signal; and an emitter configured to emit the amplified electromagnetic signal in proximity to living cells. DNA from a pathogen is amplified using PCR, purified, and serially diluted. Electromagnetic signals from the diluted DNA are received, and optionally stored. The receive signal is amplified and emitted in proximity to living cells, to produce under selected circumstances, a cytopathic effect.

### CROSS REFERENCE TO RELATED APPLICATION

The present application is a non-provisional of U.S. Provisional Application No. 62/020,796, filed Jul. 3, 2014, and U.S. Provisional Application No. 62/039,046, filed Aug. 19, 2014, each of which is expressly incorporated herein by reference it its entirety.

### FIELD OF THE INVENTION

The present invention relates to the field of electromagnetic field interaction with biological systems.

### BACKGROUND OF THE INVENTION

Each of the references cited herein are expressly incorporated herein by reference, for their teaching of the state of the art, including both techniques and specifics of technology, aspects of the present technology not expressly recited, and support for the written description for the claims presented herein, enablement for persons of ordinary skill in the art to practice the invention, and otherwise to provide disclosure.

The relationship of electromagnetic signals and biological systems is well established, for certain applications. For example, biological cells maintain different concentrations of intracellular ions than in the extracellular environment. This results in an electrical potential, the Nernst potential, at the cell membrane. Cells are able to modulate conductivity through membrane pores or transport proteins, resulting in electrical currents and corresponding magnetic fields. Organs of multicellular organisms typically communicate through electrical signals, and nervous tissue in particular exploits ion conductivity to convey information. The range of these potentials is typically below 100 mV, though in the case of some organisms, such as electric eels, 600V potentials are possible. The frequency of these signals is typically below 3 Hz, though the frequency spectrum may include components several orders of magnitude greater.

It is well known that various molecules, biological and otherwise, have bonds which form and break, with corresponding electrochemical activity. The range of potentials involved in covalent bonds is up to about 20 eV, though when including ionic bonds, the bonding energy extends through zero to negative (repulsive) levels. The frequency (and corresponding wavelength) of an electromagnetic wave corresponds to its energy, and therefore the range of emissions available from chemical environments in the environment ranges from ultraviolet and beyond to ultra-low frequencies, less than 100 Hz.

Disorganized energy emission or absorption represents noise, and such noise in equilibrium has no net emission or absorption of energy. However, above absolute zero temperature, various forms of energy are available, and can be emitted, with a corresponding reduction in temperature. Even at static temperature, and no net input of energy, a system may emit electromagnetic energy, for example a glowstick (neglecting possible exothermic reactions).

Thus, a simplistic analysis of the laws of thermodynamics to conclude that a system to which appears to be at equilibrium, and which appears to have no net receipt of energy or decrease in temperature, could not appear to emit electromagnetic radiation, is not always accurate and correct. Rather, especially at very low energy levels, one must move beyond analysis of appearances, to a rigorous energy balance including all forms of energy, in order to understand the full system state and transition.

With this in mind, a further appreciation of a large number of reports and analysis of electromagnetic signals emitted from apparently stable systems is available. That is, if one presumes that a measured electromagnetic signal obeys the laws of thermodynamics, then the molecular and chemical interactions within the system can be analyzed as the source of the electromagnetic signal.

There have been some reports of effects of external electromagnetic signals, even those of low frequency, and therefore of “non-ionizing” level, on complex biological systems. For example, there are hypotheses that 60 Hz signals from high tension power lines cause various diseases.

Other reports have suggested that electromagnetic signals, even well below the microwave energy level, can have biological effect. Indeed, there is some recognition by the US Federal Communications Commission that various radio devices must be limited in their radio frequency emissions, to avoid adverse health effects. However, the model employed is generally based on a thermal model, wherein the heating effect of the radio frequency energy on a user is calculated.

However, while the art suggests a range of bio-electromagnetic signal interactions, even at low frequencies, and ultra-low frequencies (e.g., in a range of 1 Hz-1 MHz), the effects are not well understood, and often denied, leading to difficulties in formulating and testing hypotheses and therefore producing useful results.

U.S. Pat. Nos. 6,150,812, and 7,477,053, US patent application 20040222789, and EP 2239586 A1, each of which is expressly incorporated herein by reference, relate to measurement of low frequency electromagnetic signals. The motion of the electrons within a single isolated atom or molecule generates electromagnetic fields which can be detected external to the boundaries of the atom or molecule. The magnitude and frequency of such external fields depends mainly upon the following factors: (i) the angular momentum of the electron as it spins on its axis (=electron spin angular momentum), (ii) the angular momentum of the electron as it moves in quasicircular orbital paths around the nucleus (=electron orbital momentum), (iii) the quantized energy states of the electron orbital paths and angular spin velocities, (iv) interactions between intraatomic and intramolecular electron motions as governed by Lenz's law, (v) rate of individual transitions between quantized energy states and the frequency of transitional events, (vi) interactions between electron orbital and spin angular moments and nuclear magnetic moments, and (vii) intensity, frequency and direction of externally imposed magnetic fields. Other than bonding interactions, nuclei also have spin, linear and angular momentum, as well as interactions with nearby nuclei and electrons. The electromagnetic fields generated by electron motion within atoms or molecules are accompanied by the simultaneous emission of photons whose energies are characteristic of the frequencies of the associated intraatomically- or intramolecularly-generated external electromagnetic fields. The range of atomic and molecular electromagnetic frequencies extends from microwave and even lower-frequency energies, up to ultraviolet and even higher-frequency energies. Of interest are the lower energy interactions, which are typically in the “conduction” valence band of linked atoms, spins and motions of atoms and molecules, but typically not representing changes in electron states between lower valence states, corresponding to higher energies. It is noted that through resonance and multiple photon capture, relatively lower energy states may be converted into higher ones. In particular, large numbers of interacting atoms, such as water molecules which solvate a biological macromolecule, and in the aggregate possess a significant amount of energy, with no one molecule having high energy. However, under some conditions, there can be a coordinated transfer of energy. Indeed, biological macromolecules in the form of enzymes (catalysts) typically serve to concentrate available energy in the medium at an active site to supply an activation energy to achieve a transition state to permit a biochemical reaction to proceed. Intraatomic changes in electron position, energy state, acceleration, deceleration, in addition to various interatomic interactions are observable through associated electromagnetic fields.

Detection of low frequency electromagnetic waves and phenomena is facilitated through detection of the associated B field, for example with a conductive loop or coil (solenoid, SQUID, Hal effect sensor), rather than an H field sensor (antenna, microstrip, etc.).

It is generally believed that no net magnetic field at low (e.g., 0 to <10,000 Hz) frequency can be recorded, except over very short intervals, from macroscopic aggregates of atoms or molecules at rest in their ground state. This is because the magnetic moments of the individual atoms or molecules in such aggregates will on average find orientations whose resultant external magnetic field intensities are for all practical purposes zero. However, if the molecules or their aggregates are not at rest, are not in their ground state, or are subject to an external coercive agency, then external low frequency signals can be recorded. It is noted that the presumptions of “rest” and ground state are violated above 0K, at least to some extent. Further, the presence of chemical energy (high energy bonds and alternate possible bonds of lower energy) and organizations of molecules in other than a highest entropy state, also violate the presumptions required for an absence of low frequency magnetic emissions. Finally, environmental electromagnetic signals, which can be extremely hard to completely filter, and therefore, obtaining a condition that represents an absence of external coercive force is nearly impossible. Therefore, while under “ideal” conditions, one might not expect to see low frequency electromagnetic signals emitted from seemingly homogeneous solutions in which no chemical reaction is apparently occurring, ideal conditions may be difficult to achieve. Electromagnetic signal emissions, therefore may be due to “rare” conditions, such as highly dilute compositions, free radical excited molecules, or the like.

On the other hand, theory does not hold that it is difficult to influence a sample with externally applied electromagnetic fields, and indeed electromagnetic fields are well known to interact with ionic solutions, dipole molecules or nuclei with magnetic moments, etc.

Various studies have been conducted seeking to determine the biological effects of low frequency electromagnetic signals. A review of a portion of the literature reveals that few researchers have carefully considered and controlled for information that may be contained within the low frequency electromagnetic signals, and rather presume that the effect is based on or available from sinusoidal waves or intermittent sinusoidal waves. Even those that consider the source or information contained within the low frequency electromagnetic signals have to date not fully considered resonances, information communication, and implications for informational biopolymers, such as DNA.

C F Blackman, “Treating cancer with amplitude-modulated electromagnetic fields: a potential paradigm shift, again?”, British Journal of Cancer (2012) 106, 241-242. doi:10.1038/bjc.2011.576 www.bjcancer.com discusses various biological effects of electromagnetic fields (EMFs). Barbault et al (2009) describes how they obtained the specific frequencies for different tumor diagnoses, which are then used in the amplitude-modulated (AM)-EMF treatment of those patients to stabilize the disease beyond normal expectations. Costa et al (2011) reported surprising clinical benefits from using the specific AM-EMF signals to treat advanced hepatocellular carcinoma, stabilizing the disease and even producing partial responses up to 58 months in a subset of the patients. Zimmerman et al have examined the growth rate of human tumor cell lines from liver and breast cancers along with normal cells from those tissues exposed to AM-EMF. Reduced growth rate was observed for tumor cells exposed to tissue-specific AM-EMF, but no change in growth rate in normal cells derived from the same tissue type, or in tumor or normal cells from the other tissue type. The growth rate inhibitory response was field-strength (SAR) and exposure-time dependent. In ancillary tests, they observed reduction in gene expression and increases in mitotic spindle dysfunction only for the AM-EMF exposure that reduced the cell growth rate.

Bawin et al, 1975, with independent replication by Blackman et al, 1979, demonstrated that biological effects could be caused by certain AM frequencies on a carrier wave but not other frequencies. See also Adey, 1992; Blackman, 1992. This growing collection of reports demonstrating AM-EMF-induced biological effects led to recognition by national and international authorities that this modality needed to be considered in hazard evaluation, in addition to field-induced heating as a cause for health concern. The National Council on Radiation Protection and Measurements (1986) recommended a reduction in the allowable exposure intensity limits for AM radiation above a certain level, and the World Health Organization (1993) explicitly acknowledged AM as a future issue to be examined in setting exposure guidelines. Barbault et al (2009) identifies relevant treatment frequencies can be seen to have direct clinical and medical relevance in determining the characteristics of a new modality that may prove useful in cancer treatment.

Zimmerman et al demonstrates the fundamental requirement for a biological ‘information content’ code (i.e., the AM spectral profile) that can affect tumor cells from the tissue of origin, while apparently being ignored by normal cells from various tissues and tumor cells from different tissues of origin. By exposing HCC cells to 27.12□MHz RF EMF sinusoidally amplitude-modulated at specific frequencies, which were previously identified in patients with a diagnosis of HCC (Barbault et al, 2009) and result in therapeutic responses in patients with HCC (Costa et al, 2011), a robust and sustained anti-proliferative effect was demonstrated. This effect was seen within SARs ranging from 0.03 to 1.0□W□kg-1. HCC-specific modulation frequencies began to hinder cell proliferation after 7 days of exposure and the anti-proliferative effect increased over a 7-week period. The anti-proliferative effect HCC-specific modulation frequencies were observed only in HCC cells, but not in breast cancer cells or normal hepatocytes. Two sets of similar modulation frequencies (breast cancer-specific and randomly chosen) within the same range (from 100□Hz to 21□kHz) did not affect the proliferation of HCC cells. Similarly, the proliferation of breast cancer cells was affected only by breast cancer-specific modulation frequencies, but neither by HCC-specific nor by randomly chosen modulation frequencies.

Modulation of the signal appears to be a critical factor in the response of biological systems to electromagnetic fields (Blackman, 2009). The amount of electromagnetic energy delivered is far too low to break chemical bonds or cause thermal effects. Several theories have been put forth to explain biological responses to electromagnetic fields. Some reports have shown that low levels of electromagnetic fields can alter gene expression and subsequent protein synthesis by interaction of the electromagnetic field with specific DNA sequences within the promoter region of genes (Blank and Goodman, 2008; Blank and Goodman, 2009). Such changes have been demonstrated in the family of ‘heat shock’ proteins that function in the cell stress response (Blank and Goodman, 2009). Zimmerman et al. interrogated gene expression changes in cells exhibiting decreased cell proliferation, using high-throughput sequencing technologies to sequence the cells' cDNA. Tumor cell GI was associated with downregulation of PLP2 and XCL2 as well as with disruption of the mitotic spindle. Exposure of HCC cells to the same RF EMF modulated at slightly different modulation frequencies did not result in changes in gene expression, which demonstrates that inhibition of cell proliferation is associated with changes in gene expression levels. Very low levels of 27.12□MHz radio frequency electromagnetic fields were shown to inhibit tumor cell growth when modulated at specific frequencies.

Martin Blank, Reba Goodman, “A mechanism for stimulation of biosynthesis by electromagnetic fields: Charge transfer in DNA and base pair separation”, Journal of Cellular Physiology, Volume 214, Issue 1, pages 20-26, January 2008, DOI: 10.1002/jcp.21198 (2007), considers possible mechanisms for the biological effect of low frequency electromagnetic fields. Electrons have been shown to move in DNA, and a specific DNA sequence is associated with the response to EM fields. In addition, there is evidence from biochemical reactions that EM fields can accelerate electron transfer. Interaction with electrons could displace electrons in H-bonds that hold DNA together, leading to chain separation and (in cellular systems) initiating transcription. The effect of charging due to electron displacement on the energetics of DNA aggregation shows that electron transfer would favor separation of base pairs, and that DNA geometry is optimized for disaggregation under such conditions. Electrons in the H-bonds of both DNA and the surrounding water molecules fluctuate at frequencies that are much higher than the frequencies of the EM fields studied. The characteristics of the fluctuations suggest that the applied EM fields are effectively DC pulses and that interactions extend to microwave frequencies.

Lai and Singh 1997 found that rats acutely exposed to a 60-Hz sinusoidal magnetic field showed an increase in DNA single- and double-strand breaks in their brain cells as measured by the microgel electrophoresis assay. An increase in DNA single-strand breaks was observed after 2 hr of exposure to the magnetic field at flux density of ≦0.1 millitesla (mT), whereas an increase in double-strand breaks was observed at ≦0.25 mT. Using the microgel electrophoresis assay, Ahuja et al. (1997, 1999), Phillips et al. (1997), Svedenstal et al. (1999a, 1999b), and Zmyslony et al. (2000) have also reported an increase in DNA strand breaks in cells after magnetic field exposure. In studies by Ahuja et al. (1997, 1999), an increase in DNA single-strand breaks in human lymphocytes was observed after 1 hr of exposure to a 50-Hz magnetic field at 0.2-2 mT, whereas in the study by Phillips et al. (1997), an increase in single-strand breaks was observed in human Molt-4 cells after 24 hr of exposure to a 60-Hz magnetic field at 0.1 mT. Svedenstal et al. observed an increase in DNA double-strand breaks in brain cells of mice after 32 days of exposure to magnetic fields of 7.5 μT (Svedenstal et al. 1999a) and after 14 days of exposure at 0.5 mT (Svedenstal et al. 1999b). Zmyslony et al. (2000) reported an increase in single-strand breaks in rat lymphocytes exposed to a 50-Hz magnetic field at 7 mT in the presence of iron cations. Ivancsits et al. (2002, 2003a, 2003b) reported an increase in DNA single- and double-strand breaks in human fibroblasts intermittently (5 min on/10 min off) exposed to a 50-Hz magnetic field at 1 mT, whereas continuous exposure produced no significant effect, and therefore indicate that the interaction of magnetic fields with DNA is quite complicated and apparently depends on many factors. McNamee et al. (2002) reported no significant effect on DNA strand breaks in cerebellar cells of immature mice exposed continuously to a 60-Hz magnetic field at 1 mT for 2 hr. Miyakoshi et al. (2000) reported that a high-intensity (>50 mT) 50-Hz magnetic field had no significant effect alone, whereas it potentiated X-ray-induced DNA single-strand breaks in human glioma cells. Thus, effects of magnetic fields on DNA may depend on factors such as the mode of exposure, the type of cells studies, and the intensity and duration of exposure.

Lai and Singh 1997b found that pretreating rats with melatonin and a spin-trap compound (N-tert-butyl-α-phenylnitrone) blocked the effect of a 60-Hz magnetic field on DNA. Because melatonin and spin-trap compounds are efficient free-radical scavengers, the data suggest that free radicals play a role in the effect of the magnetic field. Singh and Lai 1998 found that acute magnetic field exposure induced the formation of DNA-protein and DNA-DNA cross-links in brain cells of rats, which could be the results of free-radical damage involving iron cations (Altman et al. 1995; Lloyd et al. 1997). Exposure to a 60-Hz magnetic field at 0.01 mT for 24 hr caused a significant increase in DNA single- and double-strand breaks. Prolonging the exposure to 48 hr caused a larger increase. This indicates that the effect is cumulative. Treatment with Trolox (Forrest et al. 1994, a vitamin E analog) or 7-nitroindazole (Kalisch et al. 1996; Moore and Bland-Ward 1996, a nitric oxide synthase inhibitor) blocked magnetic-field-induced DNA strand breaks. These data further support a role of free radicals on the effects of magnetic fields. Treatment with the iron chelator deferiprone (Fredenburg et al. 1996; Kontoghiorghes 1995) also blocked the effects of magnetic fields on brain cell DNA, suggesting the involvement of iron. Acute magnetic field exposure increased apoptosis and necrosis of brain cells in the rat. Exposure to a 60-Hz magnetic field was hypothesized to initiate an iron-mediated process (e.g., the Fenton reaction) that increases free radical formation in brain cells, leading to DNA strand breaks and cell death. This hypothesis could have an important implication for the possible health effects associated with exposure to extremely low-frequency magnetic fields in the public and occupational environments. Henry Lai and Narendra P. Singh, “Magnetic-Field-Induced DNA Strand Breaks in Brain Cells of the Rat”, Environmental Health Perspectives, v. 112, no. 6, May 2004, p. 687

Potenza L l, Cucchiarini L, Piatti E, Angelini U, Dacha M., “Effects of high static magnetic field exposure on different DNAs.”, Bioelectromagnetics. 2004 July; 25(5):352-5 disclose the effects of magnetic fields produced by permanent magnets on different DNA sources. Escherichia coli DNA, plasmid, and amplification products of different lengths were used as the magnetic field target. The in vivo assays did not reveal any DNA alterations following exposure, demonstrating the presence of cell dependent mechanisms, such as the repair system and the buffering action of the heat shock proteins DNA K/J (Hsp 70/40). In vitro assays displayed interactions between the magnetic field and DNA, revealing principally that magnetic field exposure induces DNA alterations in terms of point mutations. They speculate that the magnetic field can perturb DNA stability interacting with DNA directly or potentiating the activity of oxidant radicals. This genotoxic effect of the magnetic field, however, is minimized in living organisms due to the presence of protective cellular responses.

Ivancsits S, Diem E, Jahn O, Rüdiger H W, “Intermittent extremely low frequency electromagnetic fields cause DNA damage in a dose-dependent way.”, Int Arch Occup Environ Health. 2003 July; 76(6):431-6. Epub 2003 Jun. 12, disclose that epidemiological studies have reported an association between exposure to extremely low frequency electromagnetic fields (ELF-EMFs) and increased risk of cancerous diseases, albeit without dose-effect relationships. The validity of such findings can be corroborated only by demonstration of dose-dependent DNA-damaging effects of ELF-EMFs in cells of human origin in vitro. Cultured human diploid fibroblasts were exposed to intermittent ELF electromagnetic fields. DNA damage was determined by alkaline and neutral comet assay. ELF-EMF exposure (50 Hz, sinusoidal, 1-24 h, 20-1,000 mu T, 5 min on/10 min off) induced dose-dependent and time-dependent DNA single-strand and double-strand breaks. Effects occurred at a magnetic flux density as low as 35 mu T, being well below proposed International Commission of Non-Ionising Radiation Protection (ICNIRP) guidelines. After termination of exposure the induced comet tail factors returned to normal within 9 h. Ivancsits et al. concluded that the induced DNA damage is not based on thermal effects and arouses concern about environmental threshold limit values for ELF exposure.

Ivancsits S, Diem E, Jahn O, Rüdiger H W. “Age-related effects on induction of DNA strand breaks by intermittent exposure to electromagnetic fields.”, Mech Ageing Dev. 2003 July; 124(7):847-50 disclose that several studies indicating a decline of DNA repair efficiency with age raise the question, if senescence per se leads to a higher susceptibility to DNA damage upon environmental exposures. Cultured fibroblasts of six healthy donors of different age exposed to intermittent ELF-EMF (50 Hz sinus, 1 mT) for 1-24 h exhibited different basal DNA strand break levels correlating with age. The cells revealed a maximum response at 15-19 h of exposure. This response was clearly more pronounced in cells from older donors, which could point to an age-related decrease of DNA repair efficiency of ELF-EMF induced DNA strand breaks.

Robison J G, Pendleton A R, Monson K O, Murray B K, O'Neill K L, “Decreased DNA repair rates and protection from heat induced apoptosis mediated by electromagnetic field exposure.” Bioelectromagnetics. 2002 February; 23(2):106-12., disclose that electromagnetic field (EMF) exposure results in protection from heat induced apoptosis in human cancer cell lines in a time dependent manner. Apoptosis protection was determined by growing HL-60, HL-60R, and Raji cell lines in a 0.15 mT 60 Hz sinusoidal EMF for time periods between 4 and 24 h. After induction of apoptosis, cells were analyzed by the neutral comet assay to determine the percentage of apoptotic cells. To discover the duration of this protection, cells were grown in the EMF for 24 h and then removed for 24 to 48 h before heat shock and neutral comet assays were performed. The results demonstrate that EMF exposure offers significant protection from apoptosis (P<0.0001 for HL-60 and HL-60R, P<0.005 for Raji) after 12 h of exposure and that protection can last up to 48 h after removal from the EMF. In this study they further demonstrate the effect of the EMF on DNA repair rates. DNA repair data were gathered by exposing the same cell lines to the EMF for 24 h before damaging the exposed cells and non-exposed cells with H2O2. Cells were allowed to repair for time periods between 0 and 15 min before analysis using the alkaline comet assay. Results showed that EMF exposure significantly decreased DNA repair rates in HL-60 and HL-60R cell lines (P<0.001 and P<0.01 respectively), but not in the Raji cell line. The apoptosis results show that a minimal time exposure to an EMF is needed before observed effects. This may explain previous studies showing no change in apoptosis susceptibility and repair rates when treatments and EMF exposure were administered concurrently.

Zhou J, Yao G, Zhang J, Chang Z, “CREB DNA binding activation by a 50-Hz magnetic field in HL60 cells is dependent on extra- and intracellular Ca(2+) but not PKA, PKC, ERK, or p38 MAPK”, Biochem Biophys Res Commun. 2002 Aug. 30; 296(4):1013-8, disclose that, to investigate the possible mechanism of gene transcription changes induced by magnetic field (MF), they examined the DNA binding behavior of the transcription factor cyclic-AMP responsive element binding protein (CREB) in HL60 cells after exposure to a 0.1 mT 50-Hz extremely low frequency (ELF) sinusoidal MF by a gel shift assay. Magnetic field induced a time-dependent activation of CREB binding. The complex formation increased shortly after MF exposure for 10 min, reaching a peak level after 1 h, and then recovered to basal level at 4 h after exposure. A novel MF-induced ATF2/ATF2 homodimer formation was observed after MF exposure for 30 min, 1, and 2 h. Furthermore, They found that the MF-induced increase of CREB DNA binding in HL60 cells was dependent on both extracellular and intracellular Ca(2+) but not PKA, PKC, ERK, or p38 MAPK by using various pathway inhibitors. These data indicate that MF exposure activates CREB DNA binding through calcium-related signal transduction pathways under the experimental conditions.

Singh N, Lai H, “60 Hz magnetic field exposure induces DNA crosslinks in rat brain cells,” Mutat Res. 1998 May 25; 400(1-2):313-20 disclose that, in previous research, they found an increase in DNA strand breaks in brain cells of rats acutely exposed to a 60 Hz magnetic field (for 2 h at an intensity of 0.5 mT). DNA strand breaks were measured with a microgel electrophoresis assay using the length of DNA migration as an index. In the present experiment, they found that most of the magnetic field-induced increase in DNA migration was observed only after proteinase-K treatment, suggesting that the field caused DNA-protein crosslinks. In addition, when brain cells from control rats were exposed to X-rays, an increase in DNA migration was observed, the extent of which was independent of proteinase-K treatment. However, the X-ray-induced increase in DNA migration was retarded in cells from animals exposed to magnetic fields even after proteinase-K treatment, suggesting that DNA-DNA crosslinks were also induced by the magnetic field. The effects of magnetic fields were also compared with those of a known DNA crosslink-inducing agent mitomycin C. The pattern of effects is similar between the two agents. These data suggest that both DNA-protein and DNA-DNA crosslinks are formed in brain cells of rats after acute exposure to a 60 Hz magnetic field.

Lai H, Singh N P, “Melatonin and N-tert-butyl-alpha-phenylnitrone block 60-Hz magnetic field-induced DNA single and double strand breaks in rat brain cells.” J Pineal Res. 1997 April; 22(3):152-62, discloses that In previous research, they found an increase in DNA single- and double-strand breaks in brain cells of rats after acute exposure (two hours) to a sinusoidal 60-Hz magnetic field. The present experiment was carried out to investigate whether treatment with melatonin and the spin-trap compound N-tert-butyl-alpha-phenylnitrone (PBN) could block the effect of magnetic fields on brain cell DNA. Rats were injected with melatonin (1 mg/kg, sc) or PBN (100 mg/kg, ip) immediately before and after two hours of exposure to a 60-Hz magnetic field at an intensity of 0.5 mT. They found that both drug treatments blocked the magnetic field-induced DNA single- and double-strand breaks in brain cells, as assayed by a microgel electrophoresis method. Since melatonin and PBN are efficient free radical scavengers, these data suggest that free radicals may play a role in magnetic field-induced DNA damage.

Lai H, Singh N P, “Acute exposure to a 60 Hz magnetic field increases DNA strand breaks in rat brain cells”, Bioelectromagnetics. 1997; 18(2):156-65, disclose that acute (2 h) exposure of rats to a 60 Hz magnetic field (flux densities 0.1, 0.25, and 0.5 mT) caused a dose-dependent increase in DNA strand breaks in brain cells of the animals (assayed by a microgel electrophoresis method at 4 h postexposure). An increase in single-strand DNA breaks was observed after exposure to magnetic fields of 0.1, 0.25, and 0.5 mT, whereas an increase in double-strand DNA breaks was observed at 0.25 and 0.5 mT. Because DNA strand breaks may affect cellular functions, lead to carcinogenesis and cell death, and be related to onset of neurodegenerative diseases, the data may have important implications for the possible health effects of exposure to 60 Hz magnetic fields.

De Ninno A, Congiu Castellano A, “Influence of magnetic fields on the hydration process of amino acids: vibrational spectroscopy study of L-phenylalanine and L-glutamine”, Bioelectromagnetics. 2014 February; 35(2):129-35. doi: 10.1002/bem.21823. Epub 2013 Nov. 6, disclose that attenuated total reflectance Fourier transform infrared (ATR-FTIR) spectroscopy has been used to investigate the effect of weak electromagnetic fields on the structure of L-glutamine (L-Gln) and L-phenylalnine (L-Phe) in aqueous solution. It has been found that the exposure to a DC field or a 50□Hz AC field, for a short time induces modifications in the spectra of exposed samples in agreement with the preceding observations on glutamic acid. Furthermore, the acid-base equilibrium has been investigated by using the ratio of the intensity of the deprotonated on protonated species. In the case of L-Phe, the exposure induces a measurable shift of acid dissociation constant pKa1 out of the experimental errors, while in case of L-Gln, the effect is under the limit detectable with this method. The phenomenon of the shift of the acid-base equilibrium has been connected elsewhere to modification of the water-water hydrogen bonds in the water around both the backbone and the residue (R). Here they suggest that the magnetic field modifies the water structure around the molecules and changes the hydrophobic interactions allowing the molecules of amino acids to aggregate. The differences observed in the behavior of L-Phe and L-Gln may be related to the differences in the polarity of their residues.

Podda M V, Leone L, Barbati S A, Mastrodonato A, Li Puma D D, Piacentini R, Grassi C., “Extremely low-frequency electromagnetic fields enhance the survival of newborn neurons in the mouse hippocampus”, Eur J Neurosci. 2014 March; 39(6):893-903. doi: 10.1111/ejn.12465. Epub 2013 Dec. 30, discloses that much effort has been devoted to identifying stimuli capable of enhancing adult neurogenesis, a process that generates new neurons throughout life, and that appears to be dysfunctional in the senescent brain and in several neuropsychiatric and neurodegenerative diseases. The previously reported that in vivo exposure to extremely low-frequency electromagnetic fields (ELFEFs) promotes the proliferation and neuronal differentiation of hippocampal neural stem cells (NSCs) that functionally integrate in the dentate gyms. They report studies to specifically assess the influence of ELFEFs on hippocampal newborn cell survival, which is a very critical issue in adult neurogenesis regulation. Mice were injected with 5-bromo-2′-deoxyuridine (BrdU) to label newborn cells, and were exposed to ELFEFs 9 days later, when the most dramatic decrease in the number of newly generated neurons occurs. The results showed that ELFEF exposure (3.5 h/day for 6 days) enhanced newborn neuron survival as documented by double staining for BrdU and doublecortin, to identify immature neurons, or NeuN labeling of mature neurons. The effects of ELFEFs were associated with enhanced spatial learning and memory. In an in vitro model of hippocampal NSCs, ELFEFs exerted their pro-survival action by rescuing differentiating neurons from apoptotic cell death. Western immunoblot assay revealed reduced expression of the pro-apoptotic protein Bax, and increased levels of the anti-apoptotic protein Bcl-2, in the hippocampi of ELFEF-exposed mice as well as in ELFEF-exposed NSC cultures, as compared with their sham-exposed counterparts.

Deng Y, Zhang Y, Jia S, Liu J, Liu Y, Xu W, Liu L, “Effects of aluminum and extremely low frequency electromagnetic radiation on oxidative stress and memory in brain of mice”, Biol Trace Elem Res. 2013 December; 156(1-3):243-52. doi: 10.1007/s12011-013-9847-9. Epub 2013 Oct. 26, sought to investigate the effect of aluminum and extremely low-frequency magnetic fields (ELF-MF) on oxidative stress and memory of SPF Kunming mice. Sixty male SPF Kunming mice were divided randomly into four groups: control group, ELF-MF group (2 mT, 4 h/day), load aluminum group (200 mg aluminum/kg, 0.1 ml/10 g), and ELF-MF+aluminum group (2 mT, 4 h/day, 200 mg aluminum/kg). After 8 weeks of treatment, the mice of three experiment groups (ELF-MF group, load aluminum group, and ELF-MF+aluminum group) exhibited firstly the learning memory impairment, appearing that the escaping latency to the platform was prolonged and percentage in the platform quadrant was reduced in the Morris water maze (MWM) task. Secondly are the pathologic abnormalities including neuronal cell loss and overexpression of phosphorylated tau protein in the hippocampus and cerebral cortex. On the other hand, the markers of oxidative stress were determined in mice brain and serum. The results showed a statistically significant decrease in superoxide dismutase activity and increase in the levels of malondialdehyde in the ELF-MF group (P<0.05 or P<0.01), load aluminum group (P<0.01), and ELF-MF+aluminum group (P<0.01). However, the treatment with ELF-MF+aluminum induced no more damage than ELF-MF and aluminum did, respectively. In conclusion, both aluminum and ELF-MF could impact on learning memory and pro-oxidative function in Kunming mice. However, there was no evidence of any association between ELF-MF exposure with aluminum loading.

Tofani S, Barone D, Cintorino M, de Santi M M, Ferrara A, Orlassino R, Ossola P, Peroglio F, Rolfo K, Ronchetto F, “Static and ELF magnetic fields induce tumor growth inhibition and apoptosis”, Bioelectromagnetics. 2001 September; 22(6):419-28, sought to evaluate the ability of static and extremely low frequency (ELF) Magnetic Fields (MF) to interfere with neoplastic cell function. In vitro experiments were carried out to study the role of MF characteristics (intensity, frequency, and modulation) on two transformed cell lines (WiDr human colon adenocarcinoma and MCF-7 human breast adenocarcinoma) and one nontransformed cell line (MRC-5 embryonal lung fibroblast). Increase in cell death morphologically consistent with apoptosis was reported exclusively in the two transformed cell lines. Cell-death induction was observed with MF of more than 1 mT. It was independent of the MF frequency and increased when modulated MF (static with a superimposition of ELF at 50 Hz) were used. Based on the in vitro results, four different MF exposure characteristics were selected and used to treat nude mice xenografted with WiDr cells. The treatment of nude mice bearing WiDr tumors subcutaneously. with daily exposure for 70 min to MF for 4 weeks caused significant tumor growth inhibition (up to 50%) by the end of the treatment when modulated MF were used for at least 60% of the whole treatment period and the time-averaged total MF intensity was higher than 3.59 mT. No toxic morphological changes induced by exposure were observed in renewing, slowly proliferating, or static normal cells.

Wen J, Jiang S, Chen B, “The effect of 100□Hz magnetic field combined with X-ray on hepatoma-implanted mice”, Bioelectromagnetics. 2011 May; 32(4):322-4. doi: 10.1002/bem.20646. Epub 2011 Feb. 22, describe that their previous cellular experiments demonstrated that 100□Hz magnetic field (MF) was effective at enhancing apoptosis of liver cancer cells BEL-7402 induced by X-ray irradiation. They sought to further explore the possible synergism between 100□Hz MF and X-ray in treatment of hepatoma-implanted Balb/c mice. 100□Hz MF exposure with a mean flux density of 0.7□mT was performed inside an energized solenoid coil. Six MV X-ray irradiation was generated using a linear accelerator. Tumor growth and survival of mice implanted with H22 cells were evaluated by measuring the tumor diameters and overall days of survival. Six groups treated with 100□Hz MF or X-ray alone or a combination of MF and X-ray were examined. Furthermore, the effects of different numbers of MF exposure periods on tumor growth and mice survival were examined when combined with 4□Gy X-ray. Data referring to overall survival days and tumor diameters of the above groups were compared using log-rank test and Student's t-test. The results showed that five periods of combined 100□Hz MFs and 4□Gy X-ray could significantly extend the overall days of survival and reduce the tumor size compared to MF or X-ray alone. Also, a greater number of 100□Hz MF exposure periods could further improve the survival and inhibit tumor growth in hepatoma-implanted mice when combined with 4□Gy X-ray. In conclusion, these findings suggested that 100□Hz MF could possibly synergize with 4□Gy X-ray in terms of survival improvement and tumor inhibition in hepatoma-implanted mice.

Delle Monache S, Angelucci A, Sanita P, Iorio R, Bennato F, Mancini F, Gualtieri G, Colonna R C, “Inhibition of angiogenesis mediated by extremely low-frequency magnetic fields (ELF-MFs)”, PLoS One. 2013 Nov. 14; 8(11):e79309. doi: 10.1371/journal.pone.0079309. eCollection 2013, describe that the formation of new blood vessels is an essential therapeutic target in many diseases such as cancer, ischemic diseases, and chronic inflammation. In this regard, extremely low-frequency (ELF) electromagnetic fields (EMFs) seem able to inhibit vessel growth when used in a specific window of amplitude. They sought to investigate the mechanism of anti-angiogenic action of ELF-EMFs they tested the effect of a sinusoidal magnetic field (MF) of 2 mT intensity and frequency of 50 Hz on endothelial cell models HUVEC and MS-1 measuring cell status and proliferation, motility and tubule formation ability. MS-1 cells when injected in mice determined a rapid tumor-like growth that was significantly reduced in mice inoculated with MF-exposed cells. In particular, histological analysis of tumors derived from mice inoculated with MF-exposed MS-1 cells indicated a reduction of hemangioma size, of blood-filled spaces, and in hemorrhage. In parallel, in vitro proliferation of MS-1 treated with MF was significantly inhibited. They also found that the MF-exposure down-regulated the process of proliferation, migration and formation of tubule-like structures in HUVECs. Using western blotting and immunofluorescence analysis, data was collected about the possible influence of MF on the signalling pathway activated by the vascular endothelial growth factor (VEGF). In particular, MF exposure significantly reduced the expression and activation levels of VEGFR2, suggesting a direct or indirect influence of MF on VEGF receptors placed on cellular membrane. In conclusion MF reduced, in vitro and in vivo, the ability of endothelial cells to form new vessels, most probably affecting VEGF signal transduction pathway that was less responsive to activation. These findings could not only explain the mechanism of anti-angiogenic action exerted by MFs, but also promote the possible development of new therapeutic applications for treatment of those diseases where excessive angiogenesis is involved.

Potenza L, Saltarelli R, Polidori E, Ceccaroli P, Amicucci A, Zeppa S, Zambonelli A, Stocchi V, “Effect of 300 mT static and 50 Hz 0.1 mT extremely low frequency magnetic fields on Tuber borchii mycelium”, Can J Microbiol. 2012 October; 58(10):1174-82. doi: 10.1139/w2012-093. Epub 2012 Sep. 25, present work aimed to investigate whether exposure to static magnetic field (SMF) and extremely low frequency magnetic field (ELF-MF) can induce biomolecular changes on Tuber borchii hyphal growth. Tuber borchii mycelium was exposed for 1 h for 3 consecutive days to a SMF of 300 mT or an ELF-MF of 0.1 mT 50 Hz. Gene expression and biochemical analyses were performed. In mycelia exposed to ELF-MF, some genes involved in hyphal growth, investigated using quantitative real-time polymerase chain reaction, were upregulated, and the activity of many glycolytic enzymes was increased. On the contrary, no differences were observed in gene expression after exposure to SMF treatment, and only the activities of glucose 6-phosphate dehydrogenase and hexokinase increased. The data herein presented suggest that the electromagnetic field can act as an environmental factor in promoting hyphal growth and can be used for applicative purposes, such as the set up of new in vitro cultivation techniques.

Naira B, Yerazik M, Anna N, Sinerik A, “The impact of background radiation, illumination and temperature on EMF-induced changes of aqua medium properties”, Electromagn Biol Med. 2013 September; 32(3):390-400. doi: 10.3109/15368378.2012.735206. Epub 2013 Jan. 16, sought to study the effects of extremely low frequency electromagnetic field (ELF EMF) on physicochemical properties of physiological solution at different environmental media. The existence of frequency “windows” at 4 and 8 Hz frequencies of ELF EMF having effects on heat fusion period, hydrogen peroxide (H2O2) formation and oxygen (O2) content of water solution and different dependency on temperature, background radiation and illumination was shown. Obtained data suggest that EMF-induced effect on water physicochemical properties depends on abovementioned environmental factors. As cell bathing medium is a target for biological effects of ELF EMF, the variability of experimental data on biological effects of EMF, obtained in different laboratories, can be explained by different environmental conditions of experiments, which very often are not considered adequately.

Murugan N J, Karbowski L M, Lafrenie R M, Persinger M A, “Temporally-patterned magnetic fields induce complete fragmentation in planaria”, PLoS One. 2013 Apr. 19; 8(4):e61714. doi: 10.1371/journal.pone.0061714. Print 2013, disclose that a tandem sequence composed of weak temporally-patterned magnetic fields was discovered that produced 100% dissolution of planarian in their home environment. After five consecutive days of 6.5 hr exposure to a frequency-modulated magnetic field (0.1 to 2 μT), immediately followed by an additional 6.5 hr exposure on the fifth day, to another complex field (0.5 to 5 μT) with exponentially increasing spectral power 100% of planarian dissolved within 24 hr. Reversal of the sequence of the fields or presentation of only one pattern for the same duration did not produce this effect. Direct video evidence showed expansion (by visual estimation ˜twice normal volume) of the planarian following the first field pattern followed by size reduction (estimated ˜½ of normal volume) and death upon activation of the second pattern. The contortions displayed by the planarian during the last field exposure suggest effects on contractile proteins and alterations in the cell membrane's permeability to water. During a subsequent series of unpublished experiments involving mouse B16 melanoma cells, the same exposure paradigm employed in the present study that produced dissolution of the flatworms resulted in fragmentation of the melanoma cells [28]. Within 5 hr of the exposure to the GM field there were no discernable intact cells with the cultures that had been exposed to the procedure. Visually obvious enlargement followed by shrinkage of these cells within a similar time frame was also observed.

Zhou J, Yao G, Zhang J, Chang Z, “CREB DNA binding activation by a 50-Hz magnetic field in HL60 cells is dependent on extra- and intracellular Ca(2+) but not PKA, PKC, ERK, or p38 MAPK”, Biochem Biophys Res Commun. 2002 Aug. 30; 296(4):1013-8, sought to investigate the possible mechanism of gene transcription changes induced by magnetic field (MF), they examined the DNA binding behavior of the transcription factor cyclic-AMP responsive element binding protein (CREB) in HL60 cells after exposure to a 0.1 mT 50-Hz extremely low frequency (ELF) sinusoidal MF by a gel shift assay. Magnetic field induced a time-dependent activation of CREB binding. The complex formation increased shortly after MF exposure for 10 min, reaching a peak level after 1 h, and then recovered to basal level at 4 h after exposure. A novel MF-induced ATF2/ATF2 homodimer formation was observed after MF exposure for 30 min, 1, and 2 h. Furthermore, They found that the MF-induced increase of CREB DNA binding in HL60 cells was dependent on both extracellular and intracellular Ca(2+) but not PKA, PKC, ERK, or p38 MAPK by using various pathway inhibitors. These data indicate that MF exposure activates CREB DNA binding through calcium-related signal transduction pathways under the experimental conditions.

Wolf F I, Torsello A, Tedesco B, Fasanella S, Boninsegna A, D'Ascenzo M, Grassi C, Azzena G B, Cittadini A, “50-Hz extremely low frequency electromagnetic fields enhance cell proliferation and DNA damage: possible involvement of a redox mechanism”, Biochim Biophys Acta. 2005 Mar. 22; 1743(1-2):120-9 discloses that HL-60 leukemia cells, Rat-1 fibroblasts and WI-38 diploid fibroblasts were exposed for 24-72 h to 0.5-1.0-mT 50-Hz extremely low frequency electromagnetic field (ELF-EMF). This treatment induced a dose-dependent increase in the proliferation rate of all cell types, namely about 30% increase of cell proliferation after 72-h exposure to 1.0 mT. This was accompanied by increased percentage of cells in the S-phase after 12- and 48-h exposure. The ability of ELF-EMF to induce DNA damage was also investigated by measuring DNA strand breaks. A dose-dependent increase in DNA damage was observed in all cell lines, with two peaks occurring at 24 and 72 h. A similar pattern of DNA damage was observed by measuring formation of 8-OHdG adducts. The effects of ELF-EMF on cell proliferation and DNA damage were prevented by pretreatment of cells with an antioxidant like alpha-tocopherol, suggesting that redox reactions were involved. Accordingly, Rat-1 fibroblasts that had been exposed to ELF-EMF for 3 or 24 h exhibited a significant increase in dichlorofluorescein-detectable reactive oxygen species, which was blunted by alpha-tocopherol pretreatment. Cells exposed to ELF-EMF and examined as early as 6 h after treatment initiation also exhibited modifications of NF kappa B-related proteins (p65-p50 and I kappa B alpha), which were suggestive of increased formation of p65-p50 or p65-p65 active forms, a process usually attributed to redox reactions. These results suggest that ELF-EMF influence proliferation and DNA damage in both normal and tumor cells through the action of free radical species.

McNamee J P, Bellier P V, Chauhan V, Gajda G B, Lemay E, Thansandote A, “Evaluating DNA damage in rodent brain after acute 60 Hz magnetic-field exposure”, Radiat Res. 2005 December; 164(6):791-7, disclose that, in recent years, numerous studies have reported a weak association between 60 Hz magnetic-field exposure and the incidence of certain cancers. To date, no mechanism to explain these findings has been identified. The objective of the current study was to investigate whether acute magnetic-field exposure could elicit DNA damage within brain cells from both whole brain and cerebellar homogenates from adult rats, adult mice and immature mice. Rodents were exposed to a 60 Hz magnetic field (0, 0.1, 1 or 2 mT) for 2 h. Then, at 0, 2 and 4 h after exposure, animals were killed humanely, their brains were rapidly removed and homogenized, and cells were cast into agarose gels for processing by the alkaline comet assay. Four parameters (tail ratio, tail moment, comet length and tail length) were used to assess DNA damage for each comet. For each species, a significant increase in DNA damage was detected by each of the four parameters in the positive control (2 Gy X rays) relative to the concurrent nonirradiated negative and sham controls. However, none of the four parameters detected a significant increase in DNA damage in brain cell homogenates from any magnetic-field exposure (0-2 mT) at any time after exposure. The dose-response and time-course data from the multiple animal groups tested in this study provide no evidence of magnetic-field-induced DNA damage.

Kim J, Ha C S, Lee H J, Song K, “Repetitive exposure to a 60-Hz time-varying magnetic field induces DNA double-strand breaks and apoptosis in human cells”, Biochem Biophys Res Commun. 2010 Oct. 1; 400(4):739-44. doi: 10.1016/j.bbrc.2010.08.140. Epub 2010 Sep. 15, sought to investigate the effects of extremely low frequency time-varying magnetic fields (MFs) on human normal and cancer cells. Whereas a single exposure to a 60-Hz time-varying MF of 6 mT for 30 min showed no effect, repetitive exposure decreased cell viability. This decrease was accompanied by phosphorylation of γ-H2AX, a common DNA double-strand break (DSB) marker, and checkpoint kinase 2 (Chk2), which is critical to the DNA damage checkpoint pathway. In addition, repetitive exposure to a time-varying MF of 6 mT for 30 min every 24 h for 3 days led to p38 activation and induction of apoptosis in cancer and normal cells. Therefore, these results demonstrate that repetitive exposure to MF with extremely low frequency can induce DNA DSBs and apoptosis through p38 activation. These results also suggest the need for further evaluation of the effects of repetitive exposure to environmental time-varying MFs on human health.

Buldak R J, Polaniak R, Buldak L, Zwirska-Korczala K, Skonieczna M, Monsiol A, Kukla M, Dulawa-Buldak A, Birkner E, “Short-term exposure to 50□Hz ELF-EMF alters the cisplatin-induced oxidative response in AT478 murine squamous cell carcinoma cells”, Bioelectromagnetics. 2012 December; 33(8):641-51. doi: 10.1002/bem.21732. Epub 2012 Apr. 25, disclose that sought to assess the influence of cisplatin and an extremely low frequency electromagnetic field (ELF-EMF) on antioxidant enzyme activity and the lipid peroxidation ratio, as well as the level of DNA damage and reactive oxygen species (ROS) production in AT478 carcinoma cells. Cells were cultured for 24 and 72□h in culture medium with cisplatin. Additionally, the cells were irradiated with 50□Hz/1□mT ELF-EMF for 16□min using a solenoid as a source of the ELF-EMF. The amount of ROS, superoxide dismutase (SOD) isoenzyme activity, glutathione peroxidase (GSH-Px) activity, DNA damage, and malondialdehyde (MDA) levels were assessed. Cells that were exposed to cisplatin exhibited a significant increase in ROS and antioxidant enzyme activity. The addition of ELF-EMF exposure to cisplatin treatment resulted in decreased ROS levels and antioxidant enzyme activity. A significant reduction in MDA concentrations was observed in all of the study groups, with the greatest decrease associated with treatment by both cisplatin and ELF-EMF. Cisplatin induced the most severe DNA damage; however, when cells were also irradiated with ELF-EMF, less DNA damage occurred. Exposure to ELF-EMF alone resulted in an increase in DNA damage compared to control cells. ELF-EMF lessened the effects of oxidative stress and DNA damage that were induced by cisplatin; however, ELF-EMF alone was a mild oxidative stressor and DNA damage inducer. They speculate that ELF-EMF exerts differential effects depending on the exogenous conditions.

Beruto D T, Lagazzo A, Frumento D, Converti A, “Kinetic model of Chlorella vulgaris growth with and without extremely low frequency-electromagnetic fields (EM-ELF)”, J Biotechnol. 2014 January; 169:9-14. doi: 10.1016/j.jbiotec.2013.10.035. Epub 2013 Nov. 8, disclose that Chlorella vulgaris was grown in two bench-scale photobioreactors with and without the application of a low intensity, low frequency electromagnetic field (EM-ELF) of about 3 mT. Cell concentration and tendency of cells to form aggregates inside the reactor were recorded over a 30 days-time period at 0.5 L-constant medium volume in the temperature range 289-304K. At 304K, after a cultivation period of 15 days, the rate of cell death became predominant over that of growth. In the temperature range 289-299K, a two step-kinetic model based on the mitotic division and the clusterization processes was developed and critically discussed. The best-fitted curves turned out to have a sigmoid shape, and the competition between mitosis and clusterization was investigated. Without EM-ELF, the temperature dependence of the specific rate constant of the mitotic step yielded an apparent total enthalpy of 15±6 kJmol(−1), whose value was not influenced by the EM-ELF application. The electromagnetic field was shown to exert a significant effect on the exothermic clusterization step. The heat exchange due to binding between cells and liquid medium turned out to be −44±5 kJmol(−1) in the absence of EM-ELF and −68±8 kJmol(−1) when it was active. Optical microscopy observations were in agreement with the model predictions and confirmed that EM-ELF was able to enhance cell clusterization.

Amin H D, Brady M A, St-Pierre J P, Stevens M M, Overby D R, Ethier C R, “Stimulation of chondrogenic differentiation of adult human bone marrow-derived stromal cells by a moderate-strength static magnetic field”, Tissue Eng Part A. 2014 June; 20(11-12):1612-20. doi: 10.1089/ten.tea.2013.0307. Epub 2014 Feb. 7, disclose that tissue-engineering strategies for the treatment of osteoarthritis would benefit from the ability to induce chondrogenesis in precursor cells. One such cell source is bone marrow-derived stromal cells (BMSCs). The effects of moderate-strength static magnetic fields (SMFs) on chondrogenic differentiation in human BMSCs in vitro were examined. Cells were cultured in pellet form and exposed to several strengths of SMFs for various durations. mRNA transcript levels of the early chondrogenic transcription factor SOX9 and the late marker genes ACAN and COL2A1 were determined by reverse transcription-polymerase chain reaction, and production of the cartilage-specific macromolecules sGAG, collage type 2 (Col2), and proteoglycans was determined both biochemically and histologically. The role of the transforming growth factor (TGF)-β signaling pathway was also examined. Results showed that a 0.4 T magnetic field applied for 14 days elicited a strong chondrogenic differentiation response in cultured BMSCs, so long as TGF-β3 was also present, that is, a synergistic response of a SMF and TGF-β3 on BMSC chondrogenic differentiation was observed. Further, SMF alone caused TGF-β secretion in culture, and the effects of SMF could be abrogated by the TGF-β receptor blocker SB-431542. These data show that moderate-strength magnetic fields can induce chondrogenesis in BMSCs through a TGF-β-dependent pathway.

Alcaraz M, Olmos E, Alcaraz-Saura M, Achel D G, Castillo J., “Effect of long-term 50□Hz magnetic field exposure on the micronucleated polychromatic erythrocytes of mice”, Electromagn Biol Med. 2014 January; 33(1):51-7. doi: 10.3109/15368378.2013.783851. Epub 2013 Jun. 19, disclose that in recent years extremely low-frequency magnetic fields (ELF-EMF) have become widely used in human activities, leading to an increased chance of exposure to ELF-EMF. There are few reports on in vivo mammalian genotoxic effects using micronucleus (MN) assays, which generally have been used as a short-term screening system. They analyzed the possible genotoxic effect induced by long-term exposure (7, 14, 21, 28□d) of a 50□Hz ELM-MF to mice by measuring the increase in frequency of micronucleated polychromatic erythrocyte in their bone marrow (MNPCEs) and they compared it with that induced by 50□cGy of X-rays. Subsequently, they tried to reduce this chromosomal damage by administering four antioxidants substances with radioprotective capacities: dimethyl sulfoxide (DMSO), 6-n-propyl-2-thiouracil (PTU), grape-procyanidins (P) and citrus flavonoids extract (CE). The increase in micronucleated cells was higher in both physical treatments (Control□<□ELF-EMF (p□<□0.01)<X-rays (p□>□0.001)); however, the antioxidant substances only showed a genoprotective capacity against the damage induced by ionizing radiation (Ci□>□PTU□=□DMSO (p□<□0.001)>P□=□CE (p□<□0.001). The 50□Hz ELM-MF increased MNPCEs in mouse bone marrow, expressing a genotoxic capacity. Administration of antioxidant substances with radioprotective capacities known to act through the elimination of free radicals did not diminish the genotoxic effect induced by ELM-MF.

Aïda L, Soumaya G, Myriam E, Mohsen S, Hafedh A., “Effects of Static Magnetic Field Exposure on Plasma Element Levels in Rat”, Biol Trace Elem Res. 2014 Jun. 5, discloses that the magnetic fields (MFs) effect observed with radical pair recombination is one of the well-known mechanisms by which MFs interact with biological systems. SMF influenced cellular antioxidant defense mechanisms by affecting antioxidant enzymes such as superoxide dismutase (SOD), glutathione peroxidase (GPx), and catalase (CAT). However, there were insufficient reports about the effects of SMF on macro and trace elements in serum, and the results were contradictory until now. In the current study, 12 rats were divided into two groups, namely as control and exposure group (128 mT and 1 h/day during five consecutive days). The macro and trace element concentrations in serum were examined. No significant difference was observed in the sodium (Na), potassium (K), calcium (Ca), phosphorus (P), and selenium (Se) levels in rat compared to control. By contrast, exposure to SMF showed an increase in the zinc (Zn) level and a decrease in iron (Fe) concentration. Under the experimental conditions, SMF exposure cannot affect the plasma levels of macroelements, while it can disrupt Zn and Fe concentrations in rat.

Aïda L, Soumaya G, Mohsen S, Abdelmelek H., “Vitamins and glucose metabolism: the role of static magnetic fields”, Int J Radiat Biol. 2014 Jun. 5:1-23, presents a review if their own data and other data from the literature of Static Magnetic Fields (SMFs) bioeffects and, vitamins and glucose metabolism. Three main areas of investigation have been covered: static magnetic field and glucose metabolism, Static magnetic field and vitamins and role of vitamins on glucose metabolism. They conclude that the primary cause of changes in cells after incubation in external SMF is disruption of free radical metabolism and elevation of their concentration. Such disruption causes oxidative stress leading to an unsteadiness of glucose level and insulin release. Moreover, based on available data, it was concluded that exposure to SMFs alters plasma levels of vitamin A, C, D and E. these parameters can take part in disorder of glucose homeostasis and insulin release.

L. R. Yeganyan, R. E. Muradyan, F. H. Arsenyan, G. K. Bazikyan, S. N. Ayrapetyan, “Magnetically treated water at 4 Hz and 2.5 mT as a modulator of cisplatin effect on cell hydration and ouabain binding of sarcoma-180 tissue”, The Environmentalist, June 2012, Volume 32, Issue 2, pp 236-241, discloses that there are many data about the extremely low-frequency electromagnetic fields (ELF-EMF) therapeutic use, especially in the field of oncology. Recent data suggest that 4 Hz EMF having dehydration effect on tissues has a pronounced antitumor activity on sarcoma-180 in mice. It was shown that 4 Hz EMF have pronounced effects on physicochemical properties of water and water solution. Therefore, the aim of the present work was the comparative study of the modulation effect of ELF-EMF on cisplatin-induced changes cell hydration and number of ouabain receptors in membrane of sarcoma-180 tumor tissues. Tissue hydration was measured as wet weight/dry weight and expressed as a water content of g/g in dry weight. The number of 3H-ouabain receptors in membrane was counted by isotope scintillation counter. In conclusion, ELF-EMF can be a possible tool for stimulation of cisPt antitumor effect.

Santi Tofani, Marcella Cintorino, Domenico Barone, Michele Berardelli, Maria Margherita De Santi, Adriana Ferrara, Renzo Orlassino, Piero Ossola, Katia Rolfo, Flavio Ronchetto, Sergio Antonio Tripodi and Piero Tosi, “Increased mouse survival, tumor growth inhibition and decreased immunoreactive p53 after exposure to magnetic fields”, Bioelectromagnetics, Volume 23, Issue 3, pages 230-238, April 2002, discloses that the possibility that magnetic fields (MF) cause antitumor activity in vivo has been investigated. Two different experiments have been carried out on nude mice bearing a subcutaneous human colon adenocarcinoma (WiDr). In the first experiment, significant increase in survival time (31%) was obtained in mice exposed daily to 70 min modulated MF (static with a superimposition of 50 Hz) having a time average total intensity of 5.5 mT. In the second independent experiment, when mice bearing tumors were exposed to the same treatment for four consecutive weeks, significant inhibition of tumor growth (40%) was reported, together with a decrement in tumor cell mitotic index and proliferative activity. A significant increase in apoptosis was found in tumors of treated animals, together with a reduction in immunoreactive p53 expression. Gross pathology at necroscopy, hematoclinical/hematological and histological examination did not show any adverse or abnormal effects. Since pharmacological rescue of mutant p53 conformation has been recently demonstrated, the authors suggest that MF exposure may obtain a similar effect by acting on redox chemistry connected to metal ions which control p53 folding and its DNA-binding activity.

Tofani, S.; Barone, D.; Peano, S.; Ossola, P., “Anticancer activity by magnetic fields: inhibition of metastatic spread and growth in a breast cancer model”, Plasma Science, IEEE Transactions on (Volume:30, Issue: 4 Page(s): 1552-1557) (August 2002) 10.1109/TPS.2002.804209, discloses that the possibility that magnetic fields (MFs) induce anticancer activity in vivo has been investigated by using a highly metastatic human cancer model transplanted in immunoincompetent mice (CD-1, nu-nu). The nude mice, bearing a subcutaneous human breast tumor (MDA-MB-435), were exposed for 70 min daily, for six consecutive weeks, to modulated MF (static with a superimposition of extremely low-frequency fields at 50 Hz) having a time-average total intensity of 5.5 mT. A positive control group was treated with a chemotherapeutic agent (cyclophosphamide). Neither MF nor cyclophosphamide significantly reduced the total number of pulmonary metastases. Both treatments induced a significant inhibition on spread and growth of intermediate (10-100 cells) and large (>100 cells) lung metastases compared with the MF sham-treatment. The inhibition induced by the MF was significantly greater than that observed in mice treated with cyclophosphamide. Gross pathology at necroscopy, hematoclinical/hematological, and histological examination did not show any toxic or abnormal effects.

Tofani S, Barone D, Cintorino M, de Santi M M, Ferrara A, Orlassino R, Ossola P, Peroglio F, Rolfo K, Ronchetto F., “Static and ELF magnetic fields induce tumor growth inhibition and apoptosis, Bioelectromagnetics. 2001 September; 22(6):419-28, discloses that the ability of static and extremely low frequency (ELF) Magnetic Fields (MF) to interfere with neoplastic cell function has been evaluated. In vitro experiments were carried out to study the role of MF characteristics (intensity, frequency, and modulation) on two transformed cell lines (WiDr human colon adenocarcinoma and MCF-7 human breast adenocarcinoma) and one nontransformed cell line (MRC-5 embryonal lung fibroblast). Increase in cell death morphologically consistent with apoptosis was reported exclusively in the two transformed cell lines. Cell-death induction was observed with MF of more than 1 mT. It was independent of the MF frequency and increased when modulated MF (static with a superimposition of ELF at 50 Hz) were used. Based on the in vitro results, four different MF exposure characteristics were selected and used to treat nude mice xenografted with WiDr cells. The treatment of nude mice bearing WiDr tumors subcutaneously. with daily exposure for 70 min to MF for 4 weeks caused significant tumor growth inhibition (up to 50%) by the end of the treatment when modulated MF were used for at least 60% of the whole treatment period and the time-averaged total MF intensity was higher than 3.59 mT. No toxic morphological changes induced by exposure were observed in renewing, slowly proliferating, or static normal cells.

F P Costa, A C de Oliveira, R Meirelles, M C C Machado, T Zanesco, R Surjan, M C Chammas, M de Souza Rocha, D Morgan, A Cantor, J Zimmerman, I Brezovich, N Kuster, A Barbault and B Pasche, “Treatment of advanced hepatocellular carcinoma with very low levels of amplitude-modulated electromagnetic fields”, British Journal of Cancer (2011) 105, 640-648. doi:10.1038/bjc.2011.292 www.bjcancer.com, discloses that a single-group, open-label, phase I/II study was performed to assess the safety and effectiveness of the intrabuccal administration of very low levels of electromagnetic fields amplitude modulated at HCC-specific frequencies in 41 patients with advanced HCC and limited therapeutic options. Three-daily 60-min outpatient treatments were administered until disease progression or death. Imaging studies were performed every 8 weeks. The primary efficacy end point was progression-free survival 6 months. Secondary efficacy end points were progression-free survival and overall survival. Treatment was well tolerated and there were no NCI grade 2, 3 or 4 toxicities. In all, 14 patients (34.1%) had stable disease for more than 6 months. Median progression-free survival was 4.4 months (95% CI 2.1-5.3) and median overall survival was 6.7 months (95% CI 3.0-10.2). There were three partial and one near complete responses. They conclude that treatment with intrabuccally administered amplitude-modulated electromagnetic fields is safe, well tolerated, and shows evidence of antitumour effects in patients with advanced HCC.
U.S. Pat. Nos. 7,081,747, 6,724,188, 7,412,340, 4,031,462, 4,095,168, 4,365,303, 4,682,027, 4,692,685, 4,751,515, 4,822,169, 5,113,136, 5,254,950, 5,305,751, 5,339,811, 5,343,147, 5,446,681, 5,458,142, 5,465,049, 5,506,500, 5,508,203, 5,541,413, 5,574,369, 5,583,432, 5,656,937, 5,696,691, 5,734,353, 5,752,514, 5,789,961, 5,944,782, 5,952,978, 5,955,400, 5,959,548, 6,020,782, 6,028,558, 6,084,399, 6,136,541, 6,142,681, 6,150,812, 6,159,444, 6,196,057, 6,204,821, 6,232,455, 6,285,249, 6,294,911, 6,320,369, 6,323,632, 6,411,108, 6,516,281, 6,541,978, 6,724,188, 6,760,674, 6,815,949, 6,885,192, 6,952,652, 6,995,558, 7,081,747, 2002/0158631, 2003/0016010, 2004/0038937, 2003/0070604, 2004/0174154, 2004/0183530, 2004/0222789, 2005/0030016, 2005/0176391, 2006/0078998, 2009/0035757, 2009/0111159, EP0060392, WO-87-02981, WO-91-13611, WO-91-14181, WO-94-17406, WO-99-54731, WO-00-01412, WO-00-17637, WO-00-17638, WO-03-83439, WO-03-102566, WO-05-036131, EP222859, EP1112748, FR2700628, FR2811591, FR05050405, FR2894673, PCT/FR94/00791, WO9417406, PCT/FR94/00079, PCT/FR99/00908, PCT/FR99/02269, PCT/FR99/02270, WO9954731, PCT/FR99/00915, WO9954731, WO0001412, WO200017637, WO200017638, PCT/FR01/02170, PCT/FR01/02172, WO0204958, WO02004067, PCT/FR2005/050405, WO2005119271.
“Direct Nanoscale Conversion of Bio-Molecular Signals Into Electronic Information” DARPA Defense Sciences Office, 2 pages, www.darpa.mil/dso/thrust/biosci/moldice.htm.
“Engineered Bio-Molecular Nano-Devices/Systems (Moldice)” DARPA Defense Sciences Office, 1 page, www.darpa.mil/dso/thrust/biosci/moldice.htm.
“The First International Workshop on TFF; What is Biophysics Behind?”, Abstract Booklet, Jun. 15, 1996, 18 pages, www.biophysics.nl/idras.htm.
Adey R. 1997. Jim Henry's world revisited—environmental “stress” at the psychophysiological and the molecular levels. Acta Physiol Scand 640(suppl):176-179.
Adey R W (1992) Collective properties of cell membranes. In Interaction Mechanisms of Low-level Electromagnetic Fields in Living Systems, Norden B, Ramel C (eds) pp 47-77. Oxford University Press: Oxford; New York
Ahuja Y R, Bhargava A, Sircar S, Rizwani W, Lima S, Devadas A H, et al. 1997. Comet assay to evaluate DNA damage caused by magnetic fields. In: Proceedings of the International Conference on Electromagnetic Interference and Compatibility, 3-5 Dec. 1997, Hyderabad, India. Washington, D C: Institute of Electrical and Electronics Engineers, 273-276.
Ahuja Y R, Vijayashree B, Saran R, Jayashri E L, Manoranjani J K, Bhargava S C. 1999. In vitro effects of low-level, low-frequency electromagnetic fields on DNA damage in human leucocytes by comet assay. Indian J Biochem Biophys 36:318-322.
Aïssa et al., “Transatlantic Transfer of Digitized Antigen Signal by Telephone Link”, Digi Bio-FASEB 97, digibio.com/cgi-bin/node.pl?lg=us&nd=n4.sub.--3.
Aïssa, et al., Molecular signaling at high dilution or by means of electronic circuitry: Journal of Immunology, 146A, 1994
Altman S A, Zastawny T H, Randers-Eichhorn L, Cacciuttolo M A, Akman S A, Dizdaroglu M, et al. 1995. Formation of DNA-protein cross-links in cultured mammalian cells upon treatment with iron ions. Free Radic Biol Med 19:897-902.
Atkins, P. W., “Rotational and Vibrational Spectra,” Physical Chemistry, 1990, pp. 458-497, Oxford University Press, Oxford, UK.
Barbault A, Costa F P, Bottger B, Munden R F, Bomholt F, Kuster N, Pasche B (2009) Amplitude-modulated electromagnetic fields for the treatment of cancer: discovery of tumor-specific frequencies and assessment of a novel therapeutic approach. J Exp Clin Cancer Res 28: 51-60
Bassett C A (1985) The development and application of pulsed electromagnetic fields (PEMFs) for ununited fractures and arthrodeses. Clin Plast Surg 12: 259-277
Bawin S M, Kaczmarek L K, Adey W R (1975) Effects of modulated VHF fields on the central nervous system. Ann N Y Acad Sci 247: 74-81
Beauvais, Francis (2007) L'Âme des Molécules—Une histoire de la mémoire de l'eau, Coll. Mille-Mondes [2], Ed. Lulu.com, Text in French, ISBN 978-1-4116-6875-1.
Beckman K B, Ames B N. 1997. Oxidative decay of DNA. J Biol Chem 272:19633-19636.
Belon, P.; Cumps, J.; Ennis, M.; Mannaioni, P. F.; Sainte-Laudy, J.; Roberfroid, M.; Wiegant. (1999). “Inhibition of human basophil degranulation by successive histamine dilutions: Results of a European multi-centre trial”. Inflammation Research 48: 17. DOI:10.1007/s000110050376. PMID 10350142.
Benveniste en mémoire, La chronique d'Eric Fottorino, Eric Fottorino, Le Monde 6 octobre 2004. Experiments past and future Some remarks on the Memory of Water Controversy
Benveniste et al., “A Simple and Fast Method for in Vivo Demonstration of Electromagnetic Molecular Signaling (EMS) via High Dilution or Computer Recording”, FASEB Journal, vol. 13, p. A163, 1999.
Benveniste et al., “Digital Biology: Specificity of the Digitized Molecular Signal”, FASEB Journal, vol. 12, p. A412, 1998, digibio.com/cgi-bin/node.pl?lg=us&nd=n4.sub.--2.
Benveniste et al., “Specific Remote Detection of Bacteria Using an Electromagnetic/Digital Procedure”, FASEB Journal, vol. 13, p. A852, 1999, digibio.com/cgi-bin/node.pl?lg=us&nd=n4.sub.--12.
Benveniste, “Effets biologiques des hautes dilutions et transmission electromagnetique du signal moleculairs”, Centre INSERM-Clamart, 32 rue des Carnets, 94140 Clamart, France.
Benveniste, et al, “A simple and fast method for in vivo demonstration of electromagnetic molecular signaling (EMS) via high dilution or computer recording”, Environmental Sciences and Pathology (162.7-162.10).
Benveniste, et al, “Digital biology” specificity of the digitized molecular signal, Cardiac Function and Dynamics (2392-2393).
Benveniste, et al, “QED and Digital Biology”, Rivista di Biologia, Biology Forum 97 (2004), pp. 169-172.
Benveniste, et al, “The molecular signal is not functional in the absence of “informed” water”, Environmental Sciences and Pathology (1621-162.10).
Benveniste, et al, “Transfer of the molecular signal by electronic amplification”, Electromagnetic Fields and Radiation (2301-2306).
Benveniste, et al., “Digital Recording/Transmission of the Cholinergic Signal”, DigiBio--FASEB 96, digibio.com/cgi-bin/node.pl?lg=us&nd=n4.sub.--4.
Benveniste, et al., “Electronic transmission of the cholinergic signal”, FASEB Journal, A683, 1995.
Benveniste, et al., “Transfer of molecular signals via electronic circuitry”, FASEB Journal, A602, 1993.
Benveniste, J. (1988). “Dr Jacques Benveniste replies” (PDF). Nature 334: 291. DOI:10.1038/334291a0. geocities.yahoo.com.br/criticandokardec/benveniste02.pdf. Benveniste, J., “From ‘Water Memory’ effects To ‘Digital Biology’ . . . —Understanding Digital Biology”, 4 pages, www.digibio.com/cgi-bin/node.pl?nd=n3>, Jun. 14, 1998.
Benveniste, J., “From ‘Water Memory’ effects To ‘Digital Biology’ . . . —Understanding Digital Biology”, 4 pages, www.digibio.com/cgi-bin/node.pl?nd=n3, Jun. 14, 1998.
Benveniste, J., “Molecular Signaling, What Is So Unacceptable for Ultra-Orthodox Scientists?”, 2 pages, www.digibio.com/cgi-bin/node.pl?nd=n5.
Benveniste, J., Davenas, E. & A. Spira (1991) Comptes Rendus de l'Académie des Sciences, January.
Benveniste, J., et al., “Transfer of the molecular signal by electronic amplification”, FASEB Journal, A398, 1994.
Benveniste, J.; Ducot, B.; Spira, A. (1994). “Memory of water revisited”. Nature 370 (6488): 322. DOI:10.1038/370322a0. PMID 8047128.
Benveniste, J.; Guillonnet, Didier (1999). “III—Demonstration challenge, etc.”. DigiBio NewsLetter 1999:2 www.digibio.com/doc/n11999-2us.txt.
Benveniste, J.; P. Jurgens, W. Hsueh & J. Aïssa (1997). “Transatlantic Transfer of Digitized Antigen Signal by Telephone Link”. Journal of Allergy and Clinical Immunology—Program and abstracts of papers to be presented during scientific sessions AAAAI/AAI.CIS Joint Meeting Feb. 21-26, 1997. Poster. www.csicop.org/si/show/e-mailed_antigens_and_iridiumrsquos_iridescence/.
Benveniste, J.; P. Jurgens, W. Hsueh and J. Aïssa (Feb. 21-26, 1997). “Transatlantic Transfer of Digitized Antigen Signal by Telephone Link”. Journal of Allergy and Clinical Immunology.
Benveniste, Jacques (1993). “Molecular signaling at high dilution or by means of electronic circuitry”. Journal of Immunology 150: 146A.
Benveniste, Jacques (1994) “Transfer of the molecular signal by electronic amplification.” FASEB Journal 8:A398.
Benveniste, Jacques (1995) “Direct transmission to cells of a molecular signal via an electronic device.” FASEB Journal 9: A227
Benveniste, Jacques (1995) “Electronic transmission of the cholinergic signal.” FASEB Journal 9: A683
Benveniste, Jacques (2005) Ma vérité sur la ‘mémoire de l'eau’, Albin Michel. ISBN 2-226-15877-4
Benveniste, Jacques, “Transfer of Biological Activity by Electromagnetic Fields.” Frontier Perspectives 3(2) 1993:113-15.
Benveniste, Jacques, and Peter Jurgens. On the Role of Stage Magicians in Biological Research The Anomalist 1998 Benveniste, Jacques, et al. (2000) “Activation of human neutrophils by electronically transmitted phorbol-myristate acetate.” Medical Hypotheses 54
Benveniste, Jacques, J. Aïssa and D. Guillonnet. (1999) “A simple and fast method for in vivo demonstration of electromagnetic molecular signaling (EMS) via high dilution or computer recording.” FASEB Journal 13:A163.
Benveniste, Jacques, J. Aïssa, P. Jurgens and W. Hsueh (1998) “Digital biology: Specificity of the digitized molecular signal.” FASEB Journal 12:A412.
Benveniste, Jacques, L. Kahhak, and D. Guillonnet (1999) “Specific remote detection of bacteria using an electromagnetic/digital procedure.” FASEB 13:A852.
Benveniste, Jacques, P. Jurgens and J. Aïssa. (1996) “Digital recording/transmission of the cholinergic signal.” FASEB Journal 10:A1479
Benveniste, Jacques. “Further Biological Effects Induced by Ultra High Dilutions: Inhibition by a Magnetic Field”, In P.C. Endler, ed., Ultra High Dilution: Physiology and Physics. Dordrecht: Kluwe academic, 1994
Benveniste, Jacques. “Put a match to pyre review” Nature 396 Dec. 10, 1998
Benveniste, Jacques. “Where is the Heresy?” December 1998
Benveniste, Jacques. Electromagnetically Activated Water and the Puzzle of the Biological Signal INSERM Digital Biology Laboratory (Mar. 10, 1999)
Benveniste, Jacques. From “Water Memory” effects To “Digital Biology”
Benveniste, Jacques; Aïssa, J.; Jurgens, P.; Hsueh, W. (1993). “Transatlantic transfer of digitized Antigen signaling at high dilution”. FASEB Journal: A602. www.digibio.com/cgi-bin/node.pl?lg=us&nd=n4_{—}7.
Benvensite, et al, “Remote detection of bacteria using an electromagnetic/digital procedure”, HIV and infectious diseases (645.17-645.22).
Benzii, R., Sutera, A. and Vulpiani, A. (1981). J. Phys. A: Math. Gen. 14:L453-L457.
Bernhardt J H. 1985. Evaluation of human exposure to low frequency fields. In: AGARD Lecture Series No. 138: Impact of Proposed Radiofrequency Radiation Standards on Military Operation. Norugton, Essex, UK:Specialized Printing Service Ltd., 8-1-8-11.
Binhi, V., “An Analytical Survey of Theoretical Studies in the Area of Magnetoreception”, 11 pages, www.biomag.info/survey.htm, 1999.
Blackman C F (1992) Calcium release from nervous tissue: experimental results and possible mechanisms. In Interaction Mechanisms of Low-Level Electromagnetic Fields in Living Systems, Norden B, Ramel C (eds), pp 107-129. Oxford University Press: Oxford; New York
Blackman C F, Elder J A, Weil C M, Benane S G, Eichinger D C, House D E. (1979) Induction of calcium ion efflux from brain tissue by radio-frequency radiation: effects of modulation-frequency and field strength. Radio Sci 14(6S): 93-98
Blumenthal N C, Ricci J, Breger L, Zychlinsky A, Solomon H, Chen G C, et al. 1997. Effects of low-intensity A C and/or D C electromagnetic fields on cell attachment and induction of apoptosis. Bioelectromagnetics 18:264-272.
Brault, J., et al., “The Analysis and Restoration of Astronomical Data via the Fast Fourier Transform”, Astronomy and Astrophysics, vol. 13, No. 2, Jul. 1971, pp. 169-189.
Brigham, E., “The Fast Fourier Transform and Applications”, Prentice Hall, 1988, pp. 131-145.
Burridge, Jim (1992) “A Repeat of the ‘Benveniste’ Experiment: Statistical Analysis”, Research Report 100, Department of Statistical Science, University College London, England. (early version of Hirst et al.)
Chapeau-Blondeau, F., “Input-output gains for signal in noise in stochastic resonance”, Physics Letters A, vol. 232, pp. 41-48, Jul. 21, 1997, Elsevier Science B.V.
Chapeau-Blondeau, F., “Periodic and Aperiodic Stochastic Resonance with Output Signal-to-Noise Ratio Exceeding That At The Input”, International Journal of Bifurcation and Chaos, vol. 9, No. 1, pp. 267-272, 1999, World Scientific Publishing Company.
Cifra, Michal, Jeremy Z. Fields, and Ashkan Farhadi. “Electromagnetic cellular interactions.” Progress in biophysics and molecular biology 105.3 (2011): 223-246., Butler, Declan. “Trial draws fire.” Nature 468.7325 (2010): 743.,
Chaplin, Martin (2000-2006) Water Structure and Behavior London South Bank University
Coles, Peter (1989). “Benveniste under review”. Nature 340 (6229): 89. Bibcode 1989 Natur.340 . . . 89C. DOI:10.1038/340089b0. PMID 2739750.
Collins, J. J., Chow, C. C. and Imhoff, T. T. (1995). Nature 376:236-238.
Cooley, J. et al., “An Algorithm for the Machine Calculation of Complex Fourier Series”, Mathematics of Computation, April 1965, pp. 297-301, vol. 19, No. 90, American Mathematical Society, Providence, R.I.
Cosic, I. (1994). IEEE Transactions on Biomedical Engineering 41(12):1101-1114.
Costa F P, de Oliveira A C, Meirelles R, Machado M C, Zanesco T, Surjan R, Chammas M C, de Souza Rocha M, Morgan D, Cantor A, Zimmerman J, Brezovich I, Kuster N, Barbault A, Pasche B. (2011) Treatment of advanced hepatocellular carcinoma with very low levels of amplitude-modulated electromagnetic fields. Br J Cancer 105: 640-648
Coghlan, Andy. “DNA regenerated after apparent quantum teleporation.” New Scientist 209.2795 (2011): 8-9.,
Cumps, J.; Belon P, Cumps J, Ennis M, Mannaioni P F, Roberfroid M, Sainte-Laudy J, Wiegant F A (Received: 11 Dec. 2002 Accepted: 12 Nov. 2003 Published online: 21 Apr. 2004). “Histamine dilutions modulate basophil activation”. Inflammation Research (Birkhäuser Basel) 53 (5): 181-188. DOI:10.1007/s00011-003-1242-0. PMID 15105967.
Davanipour Z, Sobel E, Bowman J D, Qian Z, Will A D. 1997. Amyotropic lateral sclerosis and occupational exposure to electromagnetic fields. Bioelectromagnetics 18:28-35.
Davenas E, Beauvais F, Amara J, et al. (June 1988). “Human basophil degranulation triggered by very dilute antiserum against IgE”. Nature 333 (6176): 816-8. DOI:10.1038/333816a0. PMID 2455231.
Davenas, E., F. Beauvais, J. Arnara, M. Oberbaum, B. Robinzon, A. Miadonna, A. Tedeschi, B. Pomeranz, P. Fortner, P. Belon, J. Sainte-Laudy, B. Poitevin & J. Benveniste (1988) “Human basophil degranulation triggered by very dilute antiserum against IgE”, Nature, 333(6176):816-18.
DigiBio S. A., Experimental models, From “Water Memory” effects to “Digital Biology”, digibio.com/cgi-bin/node.pl?nd=n7.
Doodley et al, Continuous exposure of rat embryos to A 1.5 G electromagnetic filed (EMF) does not affect in vitro development and viability, Electromagnetic Fields and Radiation (2301-2306).
Duhamel, P., et al., “‘Split radix’ FFT algorithm”, Electronics Letters, The Institution of Electrical Engineers, vol. 20, No. 1, Jan. 5, 1984, pp. 14-16.
Elia, V., L. A. Marrari, and E. Napoli. “Aqueous nanostructures in water induced by electromagnetic fields emitted by EDS.” Journal of thermal analysis and calorimetry 107.2 (2012): 843-851.,
Ennis, Madeleine (Received 29 Sep. 2009; revised 5 Nov. 2009; accepted 5 Nov. 2009. Available online 15 Jan. 2010). “Basophil models of homeopathy: a sceptical view”. Homeopathy (Elsevier Ltd) 99 (1): 51-56. DOI:10.1016/j.homp.2009.11.005. PMID 20129176.
Eveson R W, Timmel C R, Brocklehurst B, Hore P J, McLauchlan K A. 2000. The effects of weak magnetic fields on radical recombination reactions in micelles. Int J Radiat Biol 76:1509-1522.
Fairbairn D W, O'Neill K L. 1994. The effect of electromagnetic field exposure on the formation of DNA single strand breaks in human cells. Cell Mol Biol 40:561-567.
Farber J L. 1994. Mechanisms of cell injury by activated oxygen species. Environ Health Perspect 102(suppl 10):7-24.
Felley-Bosco E. 1998. Role of nitric oxide in genotoxicity: implication for carcinogenesis. Cancer Metast Rev 17:25-37.
Feychting M, Jonsson F, Pedersen N L, Ahlbom A. 2003. Occupational magnetic field exposure and neurodegenerative disease. Epidemiology 14:413-419.
Fiorani M, Biagiarelli B, Vetrano F, Guidi G, Dacha M, Stocchi V. 1997. In vitro effects of 50 Hz magnetic fields on oxidatively damaged rabbit red blood cells. Bioelectromagnetics 18:125-131.
Floyd R A. 1981. DNA-ferrous iron catalyzed hydroxy free radical formation from hydrogen peroxide. Biochem Biophys Res Commun 99:1209-1215.
Foletti, Alberto, et al. “Electromagnetic Information Transfer of Specific Molecular Signals Mediated through Aqueous Systems: Experimental Findings on Two Human Cellular Models.” Session 4A6Medical Electromagnetics, RF Biological Effect, MRI: 970.,
Foletti, Alberto, et al. “Electromagnetic Information Delivery as a New Perspsctive in Medicine.” Session 4P8a Medical Electromagnetics, Biological Effects: 1620.,
Foletti, Alberto, et al. “Experimental finding on the electromagnetic information transfer of specific molecular signals mediated through the aqueous system on two human cellular models.” The Journal of Alternative and Complementary Medicine 18.3 (2012): 258-261.,
Forrest V J, Kang Y-H, McClain D E, Robinson D H, Ramakrishnan N. 1994. Oxidative stress-induced apoptosis prevented by Trolox. Free Radic Biol Med 16:675-684.
Francois C, Nyuyen-Legros J, Percheron G. 1981. Topographical and cytological distribution of iron in rat and monkey brains. Brain Res 215:317-322.
Fredenburg A M, Sethi R K, Allen D D, Yokel R A. 1996. The pharmacokinetics and blood-brain-barrier permeation of the chelators 1,2 dimethyl-, 1,2 diethyl-, and 1-[ethan-1′ol]-2-methyl-3-hydroxypyridin-4-one in the rat. Toxicology 108:191-199.
Frederick, M D:W/L Associates, Ltd. Reese J A, Jostes R F, Frazier M E. 1988. Exposure of mammalian cells to 60-Hz magnetic or electric fields: analysis for DNA single-strand breaks. Bioelectromagnetics 9:237-247.
Gauger J R. 1984. Household Appliance Magnetic Field Survey. IIT Research Institute Report E O 6549-43. Arlington, Va.:Naval Electronic Systems Command.
Gerber M R, Connor J R. 1989. Do oligodendrocytes mediate iron regulation in the human brain? Ann Neurol 26:95-98.
Giuliani, Livio, et al. “Nanostructures of Water Revealed in Recent Biophysical Experiments Are They Coherent Domains of Water Predicted by the Quantum Electromagnetic Field Theory (QEMFT)?.” Session 2AP: 307.,
Glanz, J., “Sharpening the Senses with Neural ‘Noise’”, Science, vol. 277, No. 5333, Sep. 19, 1997, 2 pages, complex.gmu.edu/neural/papers/others/science97.sub.--noise.htm-1.
Gorgun, S., “Studies on the Interaction Between Electromagnetic Fields and Living Matter Neoplastic Cellular Culture.”, 22 pages, bodyvibes.com/studyl.htm.
Grundler W, Kaiser F, Keilmann F, Walleczek J. 1992. Mechanisms of electromagnetic interaction with cellular systems. Naturwissenschaften 79:551-559.
Grundler W, Keilmann F, Putterlik V, Strube D (1982) Resonant-like dependence of yeast growth rate on microwave frequencies. Br J Cancer Suppl 5: 206-208
Hakansson N, Gustaysson P, Johansen C, Floderus B. 2003. Neurodegenerative diseases in welders and other workers exposed to high levels of magnetic fields. Epidemiology 14:420-426.
Hammer, M. & W. Jonas (2004) “Managing Social Conflict in CAM Research: The Case of Antineoplastons, “Integr. Cancer Therapy”, 3(1)59-65.Full text
Hecht, Laurence. “New evidence for a non-particle view of life.” EIR 38 (2011): 72-77.,
Hirst, S. J.; Hayes, N. A.; Burridge, J.; Pearce, F. L.; Foreman, J. C. (1993). “Human basophil degranulation is not triggered by very dilute antiserum against human IgE”. Nature 366 (6455): 527. DOI:10.1038/366525a0. PMID 8255290.
Hoffman, F., “An Introduction to Fourier Theory”, 10 pages, aurora.phys.utk.edu/.about.forrest/papers/fourier/index.html.
Ingram, D. J. E., “Spectroscopy at Radio and Microwave Frequencies,” 1967, pp. 1-16, Butterworths, London, UK.
Ismael S J, Callera F, Garcia A B, Baffa O, Falcao R P. 1998. Increased dexamethasone-induced apoptosis of thymocytes from mice exposed to long-term extremely low frequency magnetic fields. Bioelectromagnetics 19:131-135.
Ivancsits S, Diem E, Jahn O, Rudiger H W. 2003a. Intermittent extremely low frequency electromagnetic fields cause DNA damage in a dose-dependent way. Int Arch Occup Environ Health 76:431-436.
Ivancsits S, Diem E, Jahn O, Rudiger H W. 2003b. Age-related effects on induction of DNA strand breaks by intermittent exposure to electromagnetic fields. Mech Ageing Dev 124:847-850.
Ivancsits S, Diem E, Pilger A, Rudiger H W, Jahn O. 2002. Induction of DNA strand breaks by intermittent exposure to extremely-low-frequency electromagnetic fields in human diploid fibroblasts. Mutat Res 519:1-13.
Ives, John (2002) “Evaluating Unusual Claims and Devices Using a Team Approach: A Case Study”, Subtle Energies & Energy Medicine, 13(1):39-59, based on Dr. Ives Keynote Address made at the Twelfth Annual ISSSEEM Conference The Co-Creation Process in Energy Medicine: A Synergy of the Sciences and the Healing Arts, Jun. 14-19, 2002
Jajte J, Zmysony M, Palus J, Dziubaltowska E, Rajkowska E. 2001. Protective effect of melatonin against in vitro iron ions and 7 mT 50 Hz magnetic field-induced DNA damage in rat lymphocytes. Mutat Res 483:57-64.
Johansen C, Olsen J H. 1998. Mortality from amyotropic lateral sclerosis, other chronic disorders, and electric shocks among utility workers. Am J Epidemiol 148:362-368.
Jonas, W. B. & J. Jacobs (1996) Healing with Homeopathy, Warner.
Jonas, W. B.; Ives, J. A.; Rollwagen, F.; Denman, D. W.; Hintz, K.; Hammer, M.; Crawford; Henry, K. (2006). “Can Specific Biological Signals be Digitized?”. The Federation of
American Societies for Experimental Biology (FASEB) Journal 20 (1): 23-28. DOI:10.1096/fj.05-3815hyp. PMID 16394263. www.fasebj.org/cgi/content/full/20/1/23.
Kalisch B E, Connop B P, Jhamandas K, Beninger R J, Boegman R J. 1996. Differential action of 7-nitroindazole on rat brain nitric oxide synthase. Neurosci Lett 219:75-78.
Katsir G, Parola A H. 1998. Enhanced proliferation caused by a low frequency weak magnetic field in chick embryo fibroblasts is suppressed by radical scavengers. Biochem Biophys Res Commun 252:753-756.
Kaufman, I. et al., “Zero-dispersion stochastic resonance in a model for a superconducting quantum interference device”, Physical Review E, vol. 57, No. 1, pp. 78-87, January 1998, The American Physical Society.
Kelley, et al, “Further studies of abnormal development of Japanese quail embryos exposed to high level pulsed magnetic fields (PMF)”, Elecromagnetic Fields and Radiation (2301-2306).
Khadir R, Morgan J L, Murray J J. 1999. Effects of 60 Hz magnetic field exposure on polymorphonuclear leukocyte activation. Biochim Biophys Acta 1472:359-367.
Kontoghiorghes G J. 1995. Comparative efficacy and toxicity of desferrioxamine, deferiprone and other iron and aluminium chelating drugs. Toxicol Lett 80:1-18.
Krause N. 1986. Exposure of people to static and time variable magnetic fields in technology, medicine, research, and public life: dosimetric aspects. In: Biological Effects of Static and Extremely Low Frequency Magnetic Fields (Bernhardt J H, ed). Munich:MMV Medizin Verlag, 57-77.
A A, Ravikumar Kurup. “Archaeal Digoxin and Creation of Cellular Plasma State-Molecular/Cellular Electromagnetic Signal Transduction.” Advanced science express 1.1 (2013).,
Kurup, Parameswara Achutha. “Archaeal Digoxin and Creation of Cellular Plasma State-Molecular/Cellular Electromagnetic Signal Transduction.” Advances in Natural Science 4.2 (2011): 42-44.,
Lai H, Horita A, Guy A W. 1993. Effects of a 60-Hz magnetic field on central cholinergic systems of the rat. Bioelectromagnetics 14:5-15.
Lai H, Singh N P. 1997a. Acute exposure to a 60-Hz magnetic field increases DNA strand breaks in rat brain cells. Bioelectromagnetics 18:156-165.
Lai H, Singh N P. 1997b. Melatonin and N-tert-butyl-α-phenylnitrone blocked 60-Hz magnetic field-induced DNA single and double strand breaks in rat brain cells. J Pineal Res 22:152-162.
Lamont, et al, “Shielded culture chamber and controlled uniaxial magnetic field generator for very low frequency (VLF) magnetic field exposure of cells during in vitro culture”, Electromagnetic Fields and Radiation (2301-2306).
Liboff, Abraham R. “Electromagnetic vaccination.” Medical hypotheses 79.3 (2012): 331-333., De Aquino, Fran. “Transmission of DNA Genetic Information into Water by means of Electromagnetic Fields of Extremely-low Frequencies.” (2012).,
Lignon, Yves (1999) “L′Homéopathie et la mémoire de l'eau”, Les dossiers scientifiques de l'étrange, Chapter 21, Michel Lafon Publishing. ISBN 2-84098-482-2. Full text in French
Lloyd D R, Phillips D W, Carmichael P L. 1997. Generation of putative intrastrand cross-links and strand breaks by transition metal ion-mediated oxygen radical attack. Chem Res Toxicol 10:393-400.
Lourencini da Silva R, Albano F, Lopes dos Santos L R, Tavares A D Jr, Felzenszwalb I. 2000. The effect of electromagnetic field exposure on the formation of DNA lesions. Redox Rep 5:299-301.
Maddox J (June 1988). “Can a Greek tragedy be avoided?”. Nature 333 (6176): 795-7. DOI:10.1038/333795a0. PMID 3133566.
Maddox, John (1988). “Waves caused by extreme dilution”. Nature 335 (6193): 760-3. DOI:10.1038/335760a0. PMID 3185705.
Maddox, John (1988). “When to believe the unbelievable”. Nature 333: 787. Bibcode 1988 Natur.333Q.787. DOI:10.1038/333787a0. PMID 3386722.
Maddox, John; James Randi and Walter W. Stewart (28 Jul. 1988). “‘High-dilution’ experiments a delusion”. Nature 334 (6180): 287-290. DOI:10.1038/334287a0. PMID 2455869.
Marino, et al, “Transient elecromagnetic fields alter growth rate of rabbit synoviocytes (HIG-82) in vitro”, Electromagnetic Fields and Radiation (2301-2306).
McNamee J P, Beller P V, McLean J R N, Marro L, Gajda G B, Thansandote A. 2002. DNA damage and apoptosis in the immature mouse cerebellum after acute exposure to a 1 mT, 60 Hz magnetic field. Mutat Res 513:121-133.
Mello Filho A C, Meneghini R. 1984. In vivo formation of single-strand breaks in DNA by hydrogen peroxide is mediated by the Haber-Weiss reaction. Biochem Biophys Acta 781:56-63.
Meneghini R. 1997. Iron homeostasis, oxidative stress, and DNA damage. Free Radic Biol Med 23:783-792.
Milgrom, Lionel (1999) “The memory of molecules”, The Independent, March 19.
Milgrom, Lionel (Mar. 15, 2001). “Thanks for the memory”. Guardian Unlimited. www.guardian.co.uk/Archive/Article/0%2C4273%2C4152521%2C00.html.
Miyakoshi J, Yoshida M, Shibuya K, Hiraoka M. 2000. Exposure to strong magnetic fields at power frequency potentiates X-ray-induced DNA strand breaks. J Radiat Res 41:293-302.
Montagnier, Luc et al., “Electromagnetic Signals Are Produced by Aqueous Nanostructures Derived from Bacterial DNA Sequences”, Interdiscip Sci Comput Life Sci, Mar. 4, 2009, pp. 81-90.
Moore P K, Bland-Ward P A. 1996. 7-Nitroindazole: an inhibitor of nitric oxide synthase. Methods Enzymol 268:393-398.
National Council on Radiation Protection and Measurements (1986) Biological Effects and Exposure Criteria for Radiofrequency Electromagnetic Fields. pp 382, NCRP Report No. 86, NCRP: Bethesda, Md.
Neuhauser, R., “Hydrogenlike Rydberg Electrons Orbiting Molceular Clusters,” Physical Review Letters, Jun. 8, 1998, pp. 5089-5092, vol. 80, No. 23, The American Physical Society, USA.
Nokazi, D., et al., “Effects of Colored Noise on Stochastic Resonance in Sensory Neurons”, Physical Review Letters, The American Physical Society, vol. 82, No. 11, Mar. 15, 1999, 4 pages.
Noonan C W, Reif J S, Yost M, Touchstone J. 2002. Occupational exposure to magnetic fields in case-referent studies of neurodegenerative diseases. Scand J Work Environ Health 28:42-48.
Oppenheim, et al., “Digital Signal Processing”, Prentice-Hall, 1975, ISBN 0-13-214635-5, pp. 87-121.
Ovelgonne, J. H.; Bol, A. W.; Hop, W. C.; Van Wijk, R (1992). “Mechanical agitation of very dilute antiserum against IgE has no effect on basophil straining properties”. Experientia 48 (5): 504-8. DOI:10.1007/BF01928175. PMID 1376282. www.ncbi.nlm.nih.gov/entrez/query.fcgi?cmd=Retrieve&db=pubmed&dopt=Abstract&list_uids=1376282.
Phillips J L, Campbell-Beachler M, Ivaschuk O, Ishida-Jones T, Haggnen W. 1997. Exposure of molt-4 lymphoblastoid cells to a 1 G sinusoidal magnetic field at 60-Hz: effects on cellular events related to apoptosis. In: 1997 Annual Review of Research on Biological Effects of Electric and Magnetic Fields from the Generation, Delivery, and Use of Electricity.
Pitkänen, M. “DNA Waves and Water.” (2011)., Giuliani, L., et al. “New Perspectives of Bioelectromagnetics in Biology and in Medicine: DNA Spectra for Diagnostic Purposes.” Journal of Physics: Conference Series. Vol. 329. No. 1. IOP Publishing, 2011.),
Pitkänen, Matti. “DNA & Water Memory: Comments on Montagnier Group's Recent Findings.” DNA Decipher Journal 1.1 (2011).,
Proakis, J. G., et al., “Advanced digital signal processing”, Maxwell MacMillan, 1992, pp. 31-57.
Reif D W, Simmons R D. 1990. Nitric oxide mediates iron release from ferritin. Arch Biochem Biophys 283:537-541.
Reiter R J. 1997. Melatonin aspects of exposure to low frequency electric and magnetic fields. In: Advances in Electromagnetic Fields in Living Systems, Vol. 2 (Lin J C, ed). New York:Plenum Press, 1-27.
Richardson D R, Ponka P. 1997. The molecular mechanisms of the metabolism and transport of iron in normal and neoplastic cells. Biochim Biophys Acta 1331:1-40.
Rothkamm K, Lobrich M. 2003. Evidence for a lack of DNA double-strand break repair in human cells exposed to very low X-ray doses. Proc Natl Acad Sci USA 100:5057-5062.
Roy S, Noda Y, Eckert V, Traber M G, Mori A, Liburdy R, et al. 1995. The phorbol 12-myristate 13-acetate (PMA)-induced oxidative burst in rat peritoneal neutrophils is increased by a 0.1 mT (60 Hz) magnetic field. FEBS Lett 376:164-166.
Ryzhkina, I. S., L. I. Murtazina, and A. I. Konovalov. “Action of the external electromagnetic field is the condition of nanoassociate formation in highly diluted aqueous solutions.” Doklady Physical Chemistry. Vol. 440. No. 2. MAIK Nauka/Interperiodica, 2011.,
Savitz D A, Checkoway H, Loomis D P. 1998. Magnetic field exposure and neurodegenerative disease mortality among electric utility workers. Epidemiology 9:398-404.
Simko M, Kriehuber R, Weiss D G, Luben R A. 1998. Effects of 50 Hz EMF exposure on micronucleus formation and apoptosis in transformed and nontransformed human cell lines. Bioelectromagnetics 19:85-91.
Simonian N A, Coyle J T. 1996. Oxidative stress in neurodegenerative diseases. Annu Rev Pharmacol Toxicol 36:83-106.
Sinal, Ya G. (1982). Theory of Phase Transitions: Rigorous Results, Pergamon Press, Oxford.
Singh N, Anand S, Rudra N, Mathur R, Behari J. 1994a. Induction of apoptosis by electromagnetic fields. In: International Proceedings of the XVI International Cancer Congress (Rao R S, Deo M G, Sanghvi L D, Mittra I, eds). Bologna, Italy:Monduzzi Editore International Proceedings Division, 545-549.
Singh N P, Graham M M, Singh V, Khan A. 1995. Induction of DNA single-strand breaks in human lymphocyte by low doses of γ-ray. Int J Radiat Biol 68:563-570.
Singh N P, Lai H. 1998. 60-Hz magnetic field exposure induces DNA crosslinks in rat brain cells. Mutat Res 400:313-320.
Singh N P, Stephens R E, Schneider E L. 1994b. Modifications of alkaline microgel electrophoresis for sensitive detection of DNA damage. Int J Radiat Biol 66:23-28.
Singh N P, Stephens R E. 1997. Microgel electrophoresis: mechanism, sensitivity and electrostretching. Mutat Res 383:167-175.
Singh N P. 1998. A rapid method for the preparation of single cells suspension from solid tissue. Cytometry 31:229-232.
Singh N P. 2000. A simple method for accurate estimation of apoptotic cells. Exp Cell Res 256:328-337.
Sobel E, Davanipour Z, Sulkava R, Erkinjuntti T, Wikstrom J, Henderson V W, et al. 1995. Occupations with exposure to electromagnetic fields: a possible risk factor for Alzheimer's disease. Am J Epidemiol 142:515-524.
Soma, R., “Noise Outperforms White Noise in Sensitizing Baroreflux Function in the Human Brain”, Physical Review Letters, vol. 91, No. 7, 4 pages, Aug. 15, 2003, The American Physical Society.
Stohs S J, Bagchi D. 1995. Oxidative mechanisms in the toxicity of metal ions. Free Radic Biol Med 18:321-326.
Suzuki Y J, Forman H J, Sevanian A. 1997. Oxidants as stimulators of signal transduction. Free Radic Biol Med 22:269-285.
Svedenstal B-M, Johanson K-L, Mattsson M-O, Paulson L-E. 1999a. DNA damage, cell kinetics and ODC activities studied in CBA mice exposed to electromagnetic fields generated by transmission lines. In Vivo 13:507-514.
Svedenstal B-M, Johanson K-L, Mild K H. 1999b. DNA damage induced in brain cells of CBA mice exposed to magnetic fields. In Vivo 13:551-552.
Tenforde T S, Kaune W T. 1987. Interaction of extremely low frequency electric and magnetic fields with humans. Health Phys 53:583-606.
Thomas, et al, “Activation of human neutrophilis by electronically transmitted phorbol-myristate acetate”, Medical Hypotheses (2000) 54(1), 33-39.
Thomas, et al., “Direct transmission to cells of a molecular signal via an electronic device”, FASEB Journal, A227, 1995.
Thomas, et al., “Modulation of Human Neutrophil Activation by “Electronic” Phorbol Myristate Acetate (PMA)”, DigiBio, www.digibio.com/cgi-bin/node.pl?lg=us&nd=n4.sub.--5.
Thomas, Y.; Schiff, M.; Belkadi, L.; Jurgens, P.; Kahhak, L.; Benveniste, J. (2000). “Activation of Human Neutrophils by Electronically Transmitted Phorbol-Myristate Acetate”. Medical Hypotheses 54 (1): 33-39. DOI:10.1054/mehy.1999.0891. PMID 10790721.
Turin, L., “A spectroscopic mechanism for primary olfactory reception”, Chemical Senses, vol. 21, No. 6, pp. 773-791.
Villain, J. (1977). J. Phys. ClO:4793-4803.
Walleczek et al, “Actue 60-hz magnetic effects on Ca2+ (Mn2+) influx in jurkat t-cells: strict dependence on cell state” Electromagnetic Fields and Radiation (2301-2306).
Weaver, J., et al., “The response of living cells to very weak electrip fields: the thermal noise limit.”, National Library of Medicine, 2 pages, Mar. 2, 1990, www.ncbi.nim.nih.gov/entrez/query.fcgi?db=PubMed&cmd=Retrieve&-list.sub.--uids=2300806&dopt=Citation.
Widom, A., et al. “Electromagnetic Signals from Bacterial DNA.” arXiv preprint arXiv:1104.3113 (2011).,
Widom, Allan, Yogendra N. Srivastava, and John Swain. “Wireless Electromagnetic Communication Systems between Bacteria in Communities.” Key Engineering Materials 543 (2013): 318-321,
World Health Organization (1993) Environmental Health Criteria 137. “Electromagnetic Fields (300 Hz to 300 GHz)”. pp 290, Geneva
Yoshikawa T, Tanigawa M, Tanigawa T, Imai A, Hongo H, Kondo M. 2000. Enhancement of nitric oxide generation by low frequency electromagnetic field. Pathophysiology 7:131-135.
Zimmerman Z W, Pennison M J, Brezovich I, Nengun Y, Yang C T, Ramaker R, Absher D, Myers R M, Kuster N, Costa F P, Barbault A, Pasche B (2012) Cancer cell proliferation is inhibited by specific modulation frequencies. Br J Cancer 106: 307-313
Zmyslony M, Palus J, Jajte J, Dziubaltowska E, Rajkowska E. 2000. DNA damage in rat lymphocytes treated in vitro with iron cations and exposed to 7 mT magnetic fields (static or 50 Hz). Mutat Res 453:89-96.

Each of the references mentioned above and below are expressly incorporated herein by reference in their entirety.

### SUMMARY OF THE INVENTION

The present technology proceeds from an understanding that biological nucleic acids contain information, which is a part of their structure. The structure, in turn, corresponds to various types of waves and resonances, which are information-coding sequence dependent.

Further, the same waves and resonances correspond to the biological nucleic acids, and their respective information sequences. Therefore, by conveying the electromagnetic signals that correspond to a biological nucleic acid, its information content can be conveyed.

The present technology is supported by data which shows that signals from highly diluted biological nucleic acids from particular sources emit electromagnetic signals, and that these signals, whether immediately amplified and presented, or recorded and amplified and presented to a specimen container which holds nucleic acid precursors, but starts without nucleic acids, results in production of the corresponding nucleic acid. Further, the signals may selectively exert toxic effects on certain cell types, but not others, which may result from for in situ formation of the nucleic acids corresponding to the signals in the cells.

It is noted that the DNA which emits electromagnetic signals typically comes from natural living sources, and therefore may include epigenetic modifications, free radical effects and adducts, and other chemical modifications that cause it to be incompletely described by its base pair sequence.

The present technology further provides a simple procedure for transducing DNA from some bacterial pathogens into living cells in culture, with induction of cytopathic effect in these cells. The actual mechanism by which this cytopathic effect, which is selectively dependent on both the source DNA being transduced, and the target cells, is not known; however, it is believed that the signals themselves are not merely representative of a biological nucleic acid, but rather the organization of the water and perhaps other solutes in the solution around the nucleic acid. Likewise, the strength of the signal implies that the source is not a single sequence of DNA, and the basis for synchronization of emissions by a plurality of emission sources is not known. The signal represents a resonance with respect to a stable arrangement of water molecules, and that when a water sample is subjected to the electromagnetic signals, the corresponding resonance is established, and in a medium where the nucleic acid precursors are present, the emitted electromagnetic signals from a first sample of biological nucleic acids can induce formation of the corresponding biological nucleic acid in another sample.

This procedure opens the way to pinpoint in pathogenic organisms some DNA sequences which play a specific role in chronic diseases, even when the pathogenic agent have not yet been identified. That is, since the signals correspond to biological DNA in a bidirectional manner, the electromagnetic signals emitted by a sample may be analyzed to yield information about biological nucleic acids within the sample, and part of this analysis may include determining the biological effect of the electromagnetic signals on cellular systems.

It is noted that present data reveals that not all DNA emits electromagnetic signals, and that DNA that emits electromagnetic signals appear to emit different signals. However, the reason for these distinctions is not yet known.

New therapeutics targeted towards these DNA sequences may be derived. For example, it may be that electromagnetic signal transduction of DNA is biologically relevant in nature, and thus that physical contact between a source DNA molecule and a targeted effector is not necessary in order to generate an observable effect. However, the paucity of prior data demonstrating this effect in the absence of specialized instruments tends to indicate that the effect is not significant in nature, and that careful capture of the signals, amplification, and repetition over a long direction, may be required in order for significant effects to be observed. One type of therapeutic regimen involves subjecting a patient or organ of a patient to electromagnetic signal emissions from a particular source corresponding to a biological nucleic acid, which may be both high intensity and prolonged duration. Another type of therapy involves administration of agents that can disrupt or interrupt the effect of the signals on a biological system. For example, various compounds may interfere with the transduction of the electromagnetic signal into a biologically active nucleic acid. A further type of therapy involves emitting a signal that interferes with an electromagnetic signal, and thus interrupts its effect. Further therapies are possible as well.

In some previous patents (U.S. Pat. No. 8,736,250; U.S. Pat. No. 8,405,379), patent applications (US 20130224788, US 20130217000, US 20130196939, US 20130143205, US 20120024701, US 20110076710, US 20110027774, US 20100323391, WO2012142568, WO2013113000), and published papers (Montagnier, Luc, et al. “Electromagnetic signals are produced by aqueous nanostructures derived from bacterial DNA sequences.” Interdisciplinary Sciences: Computational Life Sciences 1.2 (2009): 81-90., Montagnier, Luc, et al. “DNA waves and water.” Journal of Physics: Conference Series. Vol. 306. No. 1. IOP Publishing, 2011., Montagnier, Luc, et al. “Electromagnetic detection of HIV DNA in the blood of AIDS patients treated by antiretroviral therapy.” Interdisciplinary Sciences: Computational Life Sciences 1.4 (2009): 245-253.; Montagnier, Luc. “Electromagnetic signaling from DNA: a new biomarker of chronic infection.” Chinese Bulletin of Life Sciences 3 (2010): 015.,) the inventors and others have described the possibility of capturing and recording electromagnetic signals of low frequency (EMS) emitted by DNA of pathogenic viruses and bacteria. Each of the foregoing references is expressly incorporated herein by reference in its entirety.

These emissions are produced at certain dilutions of DNA in water upon excitation by lower wave frequencies of natural or artificial origin.

That is, the signals are emitted based on energy provided to the sample either from a variety of environmental sources, or a laboratory electromagnetic signal source. Prior work has also shown that agitation of the sample may be a source of energy for emissions of EMS for a period thereafter.

The EMS are believed to convey information representing the specific sequence of the DNA, since, from their digital recording, the DNA sequence can be reproduced in distant laboratories by Polymerase Chain Reaction (PCR). We describe this phenomenon as photonic transduction of DNA.

In experiments conducted by the inventors, not all DNA sequences appear to produce EMS which have known significance. In certain cases, the PCR-derived DNA amplicon was itself able to emit EMS which could be recorded and transmitted at a distance.

This is particularly the case of an amplicon derived from the 16S ribosomal DNA sequence of Borrelia burgdorferi, the agent of Lyme disease, whose PCR (947 base pairs) and nested PCR (499 base pairs) primer sequences are as follows:

Inner

| (SEQ ID NO: 1) | |
|---|---|
| BORR16S inS | 5′-CAATCYGGACTGAGACCTGC |
| and | |
| (SEQ ID NO: 2) | |
| BORR16S inAS | 5′-ACGCTGTAAACGATGCACAC. |

One aspect of the present invention describes a set of new PCR primers for detecting a 400 bp DNA sequence uniquely present in the red blood cells of HIV infected patients, whatsoever their geographical location and their ethnic origin. This 400 bp DNA sequence has not been detected in the red blood cells of HIV negative individuals. The 400 bp sequence has some sequence homology with the “Gypsy” retrotransposon sequence of human genomic DNA (e.g., 70-80%). The sequences of the primers are the following:

pRICK 1 S
(SEQ ID NO: 4)
5′- CCT GAG AAG AGA TTT AAG AAC AAA
pRICK 1 AS
5′- CCA TAT ACT GCT TCT ARY TGC T

The optimal conditions for detecting the 400 bp amplicon by PCR in red blood cells are: annealing temperature of 56 degrees Celsius, with 50 cycles of amplification (up to about 70 cycles) in a thermocycler.

However, this sequence appears to be part of the human genome, as it is detected also by the same primers in a 99% homologous sequence located in the p region of human chromosome 1 (using BLAST against a human genome databank), a region distant from that of the 237 bp sequence (located in the q region), discussed in U.S. patent application Ser. No. 13/752,003 (Montagnier), US Pat. Pub. 2013/0196939, also located in human chromosome 1 (See Example 2).

The specificity of the Borrelia burgdorferi primers was checked first on the Borrelia burgdorferi DNA and then in patients suffering of Lyme disease. In 8 Lyme patients of chronic Lyme disease of the East Coast of the USA, all showed emissions of EMS from DNA derived from their plasma according to the procedure defined in US 20120024701; see also L. Montagnier, J. Aïssa, S. Ferris, Jl. Montagnier, and C. Lavallee. “Electromagnetic Signals Are Produced by Aqueous Nanostructures Derived from Bacterial DNA Sequences” Interdiscip Sci Comput Life Sci. 1:81-90 (2009); and L Montagnier, J Aïssa, E Del Giudice, C Lavallee, A Tedeschi and G Vitiello. “DNA waves and water”. J. Phys.: Conference Series Volume 306 Number 1. 012007 (2011), Luc Montagnier, Emilio Del Giudice, Jamal Aïssa, Claude Lavallee, Steven Motschwiller, Antonio Capolupo, Albino Polcari, Paola Romano, Alberto Tedeschi, Giuseppe Vitiello, “Transduction of DNA information through water and electromagnetic waves”, Electromagn Biol Med, 2015; 34(2): 106-112, informahealthcare.com/ebm, ISSN: 1536-8378 (print), 1536-8386 (electronic), doi: 10.3109/15368378.2015.1036072 (2015), each of which is expressly incorporated herein by reference in its entirety, and from the 499 bp band (amplicon) obtained by PCR from 16S ribosomal DNA of Borrelia burgdorferi. This suggests a persistence of the microbial agent in these patients in the chronic phase of the disease, and a possible pathogenic role of the nano-structures present in the blood circulation.

The recording of the electromagnetic signals (EMS) associated with this amplicon, named BB16, has been successfully used to transduce the corresponding DNA in water tubes, according to the procedure reported in Montagnier et al., “DNA waves and water”, J. Phys.: Conference Series Volume 306 Number 1. 012007 (2011)

It was also sent over to and reproduced in a distant laboratory (Gottingen, Germany). The DNA sequence was reconstituted from water nanostructures, by using all the ingredients of PCR, using the protocol disclosed in Montagnier et al., “DNA waves and water”, J. Phys.: Conference Series Volume 306 Number 1. 012007 (2011), and US 20120024701, and U.S. 61/476,110 (“Remote Transmission of Electromagnetic Signals Inducing Nanostructures Amplifiable into a Specific DNA Sequence”, Apr. 15, 2011), which are expressly incorporated herein by reference, which show that the TAQ polymerase used in PCR was able to read and synthesize the sequence from the specific water nanostructures induced by EMS.

The characteristics of the EMS which have biological effect have not been elucidated, and statistical tests and other forms of analysis have not revealed distinct significant differences from EMS not associated with biological effects. However, the full analysis is not completed, and of course the digital sequences which represent the EMS are of course not the same.

It is therefore an object to provide a system and method for the “teleportation” of some DNA sequences by electromagnetic waves into living cells. This is evidenced in selected cases by the ability to measure DNA associated with a source of the signal in the living cells, though cytotoxicity may result in death of those cells. The DNA is not measured in cells not subject to the treatment, is dependent on the type of DNA used as a source, and occurs selectively in certain cell types.

An system is provided for inducing cytotoxicity, comprising: a receiver configured to receive an electromagnetic signal from a container, using a receiver configured to capture electromagnetic emissions from the container over a frequency range; an amplifier configured to amplify the received electromagnetic signal; and an emitter configured to emit the amplified electromagnetic signal in proximity to living cells.

A method of producing cytotoxicity is provided, comprising: amplifying DNA from a source, e.g., a pathogen, using polymerase chain reaction technology; purifying the amplified DNA; serially diluting and mixing the purified DNA in water, to generate a dilute DNA sample in a container; receiving an electromagnetic signal from the container, using a receiver configured to capture electromagnetic emissions from the container over a frequency range; optionally recording the received electromagnetic signal; amplifying the received electromagnetic signal; and emitting the amplified electromagnetic signal in proximity to living cells.

The serially diluting may comprise obtaining a portion of a prior sample, diluting the portion of the prior sample with medium containing no DNA, and mixing the diluted portion until uniform. The diluting conveniently comprise diluting 1:9, to result in 10 fold dilutions. The medium may be at least one of water, and a water-ethanol mixture. The serial dilutions are conducted over a range of, e.g., 10⁻² to 10⁻¹⁵. Typically, the 10⁻¹⁵ dilution will be negative, and may serve as a control instead of or in addition to pure medium. The signals may require solutions in excess of 10⁻² to be observed. Therefore, dilutions of 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, 10⁻¹⁸, etc. may be obtained.

The received electromagnetic signal may be obtained over a band of 1500-2000 Hz, 400-4000 Hz, 100-10,000 Hz, 20-20,000 Hz, or ≦10 Hz to ≧22 kHz. The signal may be recorded for, e.g., 6 seconds, though a range of recording times of 1, 2, 3, 4, 5, 6, 7, 8, 9, 10, 12, 15, 20, 25, 30, 04, 60, 100, 200, 400, 800, 1500, 3000, 6000, 12000, 18000, 30000, 60,000 seconds, or more, is possible.

The pathogen may comprise, for example, a Borrelia or a Ricketsiales.

Serial dilutions of the purified DNA may be analyzed for significant electromagnetic emissions by comparison with control samples that do not have DNA, and an amplitude of emissions within a band of 1500-2000 Hz is compared with control sample. Significant electromagnetic emissions may be determined by having an amplitude of emissions in a band of 1500-2000 Hz of at least 10% over a control sample.

According to one prototype embodiment, 1 the pathogen comprises Borrellia burgdorferi and the living cells comprise HL60 cells (ATCC CCL-240™), or SUM-159 cells (Flanagan L, Van Weelden K, Ammerman C, Ethier S P, Welsh J., “SUM-159PT cells: a novel estrogen independent human breast cancer model system”; Breast Cancer Res Treat. 1999 December; 58(3):193-204; Forozan F, Veldman R, Ammerman C A, Parsa N Z, Kallioniemi A, Kallioniemi O P, Ethier S P (1999) Molecular cytogenetic analysis of 11 new breast cancer cell lines. Br J Cancer 81: 1328-1334) or U937 cells (histiocytic lymphoma, ATCC CRL 1593.2™) or MCF7 cells (breast cancer, ATCC HTB-22™), each of which exhibits a cytopathic effect when exposed to EMS derived from Borrellia burgdorferi, e.g., the BB16 EMS signal.

The amplified electromagnetic signal may be emitted by transducing the amplified signal with a copper coil having 3 layers of 420 spirals of copper wire over a bobbin length of 80 mm, an internal diameter of 50 mm, and a resistance of about 6 Ohms. The amplifying may comprise amplifying over a pass band from 10 Hz to 20 kHz, with a variable output power of up to 140 W RMS. The living cells may be exposed to the amplified electromagnetic signal having a field strength of about 5 microTesla. The exposure may be for 1, 2, 3, 4, 5, 6, 7, 8, 9, 10, 11, 12, 13, 14 or more days. In the prototype, cytotoxicity was observed in 3 days, and cell death seen at 5 and was complete by 8 days.

It is believed that the signal sequence (e.g., defined by the source of the EMS), magnetic field strength and duration are the relevant factors. Due in part to the relatively low frequencies, the magnetic field is transduced through a solenoid with a hollow core, in which a sample may be placed.

When live cells are continuously exposed to the EMS for several days, e.g., the BB16 EMS signal, a test was conducted to changes in the cells. It was found that, despite the absence of Borrelia in the sample, PCR was able to produce an amplicon from the cells that included a DNA sequence identical to the DNA which was the origin of the BB16 EMS signal, which was not found in sterile controls. At the same time, there was a strong growth inhibition of the cultured tumor cells, followed by observed death of the majority of the tumor cells.

The effect appeared specific for tumor cells of various types, e.g., HL60 cells, SUM-159 cells, U937 cells, and MCF7 cells. Non-tumor cells, such as mesenchymatous stem cells, fibroblasts, activated lymphocytes from healthy blood donor were tested, and did not display the cytopathic effects, and no amplification of Borrelia DNA by PCR was observed.

Because of this differential sensitivity of neoplastic as compared to normal cells, this technology may be used as a therapy for various neoplastic diseases. According to one embodiment, an apparatus is provided for exposing small animals (whole body) to the EMS.

The apparatus comprises a solenoid having a square section that has an aperture providing a space suitable for placing two standard size plastic mice cages (290×220×140 mm), and about 80 cm long, configured to provide a magnetic field strength in a frequency range of about 20 Hz to at least 10,000 Hz of at least 5 microTesla and in some embodiments exceeding 180 milliTesla. A current of between about 2-10 Amperes (RMS) circulates in the coil, resulting in a magnetic field of 180 milliTesla. Under these conditions, no disturbing heat is released into the tunnel.

Similar to the in vitro experiment, the animals (in plastic cages devoid of metal pieces) are exposed continuously to the BB16 EMS emitted from the coil for a period of 12 days. During this 12 day exposure, the cages are taken out of the tunnel only for short term animal care.

Exposure to the same magnetic signals of healthy mice non-inoculated with tumor cells does not alter their physical behavior nor their blood cell count.

An apparatus for treatment of small animals comprises, for example, a laptop computer (e.g., a Sony laptop running Windows 8.1) which stores in digital format recorded signals derived from a PCR amplified and aqueous solution (e.g., distilled water) diluted sample of a 499 BP fragment of the 16S ribosomal DNA of the B31 strain of Borrelia burfdorferi (ATCC 35210™), or other DNAs from pathogenic bacteria. The output may be the internal digital to analog converter of the laptop, or an external device (USB connected), such as the Creative Soundbalster X-Fi HD, or X-Fi Surround 5.1 Pro. A 20× amplifier is employed, e.g., from Conrad.

Because these frequencies are similar to the human audio spectrum, advantageously, an audio amplifier may be used, which typically provide power outputs of 50-150 or higher Watts per channel, into 4 or 8 Ohms, over a range of 20 Hz-20 kHz, with less than 1% total harmonic distortion. It is believed that the relevant frequency range for the EMS extends from about 50 or 100 Hz to 2500 Hz, with peaks observed in the 1500 Hz range, and therefore electronic equipment that handles at least this range may be used.

Similarly, because the target EMS has these characteristics, audio equipment may be used to acquire and process the signals, such as so-called “sound cards” and other computer-audio interfaces. Typically, the inputs of such devices sample at about 44 kHz or 48 kHz, and therefore are above the Nyquist frequency of the signals of interest. Likewise, the digitizers have 14-16 bits or higher resolution, which is believed to be more than adequate. As discussed above, Matlab may be used as a tool to analyze the signals, but this is by no means the only available software. Other available packages include Octave, Scilab/Xcos, NumPy/Python, SciPy/Python, Julia, and R, for example.

Similarly, a device for treatment of humans may be provided, which may have characteristics similar to magnetic resonance imaging field magnets, though the field strength need not be as high as used in MRI. It is not believed that the field uniformity need by high, as is a requirement of traditional MM. Likewise, while MRI employs perturbed static fields, the present technology employs a dynamic field. Sensing coils are not required according to the present technology. The coil may encompass the entire human body, or provide localized treatment, such as the cranium. It is believed that the therapy may be intermittent, and thus continuous exposure to the EMS over several days is not required, and rather the therapy may be provided for several hours per day over a duration of days or weeks.

A device for human brain tumor treatment is provided, in which the coil is configured to surround the head of the patient. The generator of magnetic field is composed of two Helmoltz coils which are placed symmetrically close to the temporal sides of the patient's head. The magnetic field in the middle of the head is in the order of 100 mTesla (milliTesla). The helmet associating the two coils should be either fixed on a mobile stand (patient sitting down in a chair) or fixed to a wall (patient lying in a bed). In some cases, the coil and apparatus can be sufficiently portable to permit the patient to stand and walk. For example, a lithium ion battery pack may permit untethered operation for minutes to hours, while tethered operation may permit operation indefinitely. Exposure of the patient to the magnetic field is preferably continuous, to the extent feasible, and maintained until complete disappearance of the tumor upon MRI. Gaps in therapy, such as for bathing, diagnostic tests, etc, are acceptable. Other treatments (radio-therapy, chemotherapy) are preferably discontinued during the period of EMS exposure, as it is believed that actively dividing tumor cells are more sensitive to the magnetic signaling. Similarly, a whole body device may be used for treatment of tumors existing in other parts of patient's body.

The exposed living cells may be analyzed for DNA from the source by PCR using primers adapted to amplify the DNA from the pathogen, e.g., primers specific for a 16S gene of the pathogen. The effect is not limited to DNA corresponding to 16S genes. Tests may be performed comparing DNA from different sources, e.g., signals derived from different pathogens, or different target living cells. It is of course noted that the source DNA is not limited to DNA from pathogens, and DNA from other organisms, or even synthetic DNA sequences, may be employed. DNA from pathogens, however, has been found to selectively produce a cytotoxic effect on certain target cells. A differential effect on different target cells types may be determined. In some cases, the source of the DNA may by a pathogen that harbors DNA from another organism, for example, a Rickesiales is found in humans infected with HIV that carries certain human genetic sequences.

The DNA may be of various lengths, such as less than 100 bp, 150 bp, 180 bp, 200 bp, 250 bp, 300 bp, 350 bp, 400 bp, 450 bp, 500 bp, 600 bp, 700 bp, 800 bp, 900 bp, 1000 bp, or other lengths.

An analyzer may be provided to analyze an amplitude of electromagnetic emissions. The analyzer may be configured to determine whether the electromagnetic emissions exceed an amplitude threshold within a define bandwidth. The defined bandwidth may comprise 1,500 Hz to 2,000 Hz, or consist essentially of 1,500 Hz to 2,000 Hz.

These and other object will become apparent after a review of the disclosure herein, and these objects and the preferred embodiments are not intended to be limiting on the scope of the invention.

### BRIEF DESCRIPTION OF THE DRAWINGS

The FIGURE shows a schematic diagram of a system which transduces EMS from DNA and produce a cytotoxic effect in cells.

### DETAILED DESCRIPTION OF THE PREFERRED EMBODIMENTS

### Example 1

The conditions described above for remote induction of Borrelia burgdorferi 16S RNA using the BB16 recorded EMS were artificial, and could not establish that the same phenomenon could exist in nature in living cells. The present technology covers precisely the missing link between laboratory conditions and natural conditions, showing that the same process could occur in living structures.

The detailed procedure used is as follows:

1) Capture and Recording of the EMS:

The 16S ribosomal DNA partial sequence of Borrelia burgdorferi was amplified in a thermocycler (Eppendorff) at 40 cycles with an annealing temperature of 61° C.

This optimal annealing temperature was optimized on a pure DNA sample of Borrelia burgdorferi obtained from ATTC. Initial denaturation was at 95° C. for 5 minutes. Each Thermocycle included 30 seconds at 95° C., 30 seconds at 61° C. and 60 seconds at 70° C. Final extension was at 70° C. for 10 minutes.

The amplified DNA (amplicon) was separated in an agarose gel electrophoresis apparatus, and the 499 bp band was extracted from the gel by using a Qiaquick gel extraction kit (Qiagen).

The DNA concentration was adjusted to 2 ng/ml and diluted in ten-fold dilutions in 1 ml of pure water in Eppendorf plastic polyethylene conic tubes, under a laminar flow hood.

Each serial dilution was strongly shaken for 15 seconds in a Vortex shaker, before being used for the subsequent dilution. Dilutions were from 10⁻² to 10⁻¹⁵ and each tube was placed on top of a copper coil; the electric signal was recorded twice for 6 seconds each by a micro-computer, after 500× amplification and digitization by a sound card (SoundBlaster X-FI HD, Creativelabs) as previously described (US 20110027774, US 20120024701, US 20130143205, expressly incorporated herein by reference).

The signal was recorded in 2011 from Borrelia DNA using the specific primers described above, SEQ ID NO: 1 and SEQ ID NO: 2. The amplicon showed typical emission over the background in the range of 1500-2000 Hertz. The amplitude of the overall recording was measured with the custom written routines for Matlab computer software (Mathworks, Natick Mass.), which revealed a significant increase in signal above background.

The increased amplitude over the background was measured according to the formula:

% of signal power (dB/Hz)=Avg. Power from positive sample dilutions−Avg. Power of negative unfiltered dilutions×100

Average power of negative unfiltered dilutions: A result lower or equal to 10% is considered as negative.

The electromagnetic signals (EMS) of the 16S ribosomal DNA of Borrelia burgdorferi prepared according to the above method shows more than 20% increase over background (the standard error of the background being +/−2.5%), and is therefore considered a positive response.

2) EMS-Mediated Transduction of 16S BB DNA in HL60 Cells.

HL60 is a continuous cell line derived from a patient with myeloblastic leukemia (Gallagher R, Collins S, Trujillo J, et al. (1979). “Characterization of the continuous, differentiating myeloid cell line (HL-60) from a patient with acute promyelocytic leukemia”, Blood 54 (3): 713-33. PMID 288488) registered at ATCC (CCL-240™, promyeloblast cells from acute promyelocytic leukemia). HL60 Cells were grown in RPMI 16-40 medium supplemented with 10% fetal calf serum, without antibiotics, in 25 ml Falcon flasks held vertically in a 37° C. incubator with 5% CO2/air circulation. The culture medium was changed every 4 days.

Cells were transferred to an incubator containing a copper coil with the following characteristics:

bobbin length 80 mm,

internal diameter 50 mm,

R=5.93 ohms,

3 layers of 420 spirals of copper wire,

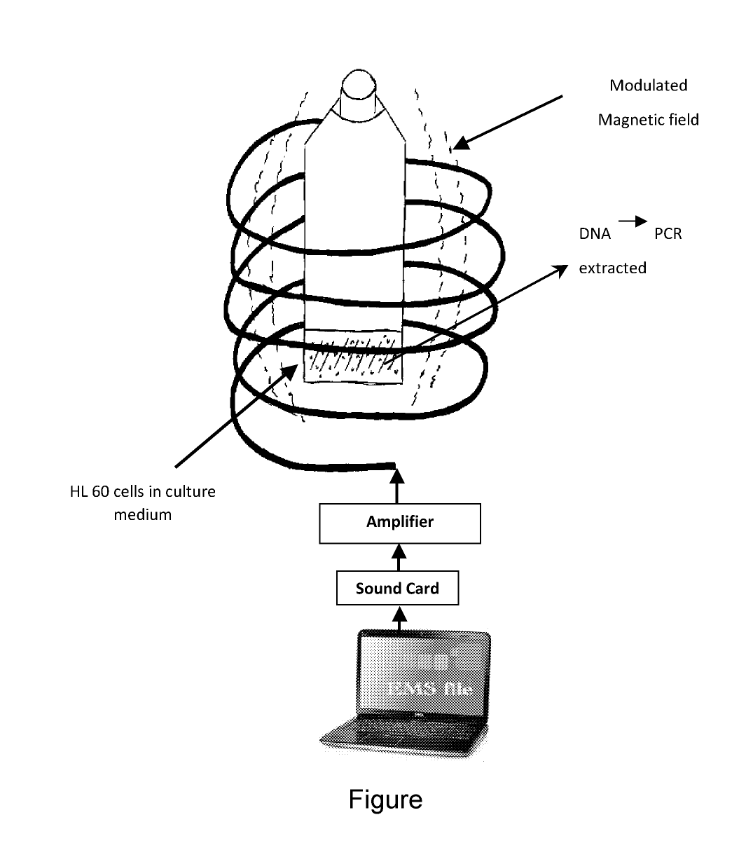

The copper coil was connected to the output of an amplifier (see FIG. 1) having the following characteristics:

pass band from 10 Hz to 20 kHz,

gain: 1 to 20

input sensitivity 250 mV

output power 140 W RMS into 8 ohms.

This amplifier was connected to a digital-analog converter (SoundBlaster sound card, Creative Labs Inc.), receiving a digital signal from a micro-computer playing the Borrelia EMS file BB16.

The cell flask (Falcon 25 mls) containing HL60 cells 1 00 000 in 8 mls of RPMI medium supplemented with 10% of fetal calf serum, was placed inside the copper coil receiving a maximal output of 4 volts from the amplifier, in order to prevent any heating of the flask. The magnetic field inside the coil under these conditions was 5 microTesla (50 gauss).

Control experiments were performed using a blank EMS file recorded from pure water, which was uncontaminated by DNA, and kept physically and magnetically isolated from EMS derived from DNA.

When the HL60 cell culture was exposed to the BB16 EMS file for 5-8 days, two effects could be observed: at day 3 following the beginning of the exposure, an inhibition of cell growth occurred, and at day 5 complete cell death was observed. (SUM-159 cells, derived from human breast cancer, as also sensitive to the Borrelia BB16 EMS.)

At day 8, the culture was interrupted and the DNA extracted from 200 microlitres of the cell suspension. An analysis by PCR (70 cycles) of the HL60 sample, using the specific 16S primers for Borrelia burgdorferi 16S RNA showed on gel electrophoresis the specific 499 bp band of 16S DNA amplicon. Centrifugation experiments (2000 rpm, 5 minutes) show that this DNA is associated with the cell pellet and is not present in the culture supernatant.

A control sample of the HL60 cells with the blank EMS file did not produce the band under the same circumstances, and cell growth inhibition and cell death were not observed, even during 8 days of culture inside the coil with the 5 microTesla signal continuously emitted.

A control sample of human macrophage cells subjected to with the BB16 EMS also did not produce the band under the same circumstances, and cell growth inhibition and cell death were not observed.

In particular, a culture of T-lymphocytes from a human healthy donor, which were activated by Phytomagglutinin (PHA) and Interleukin 2, was exposed similarly to the BB16 EMS. There was no cytopathic effect nor any sign of 16S DNA presence by PCR even after 8 days of culture.

These result would indicate, for example, that normal human differentiated cells do not have the capacity to transform the message carried by BB 16S EMS or by their derived water nanostructures into 16S DNA.

3) EMS-Mediated Transduction of Other DNAs in HL60 Cells.

In order to see if this effect was specific to the Borrelia 16SDNA, or was a general property of other amplicon-produced EMS, similar HL60 cultures were exposed to stored recordings of some other amplicons.

Indeed, cytopathic effects and specific DNA reconstitutions were obtained with EMS from the 700 bp amplicon of the 16S ribosomal DNA from a bacterium (similar to a Ricketsiales) associated with HIV infection (See US20130196939 and WO 2013/113000, expressly incorporated herein by reference), the 400 bp amplicon from the same bacterium is always associated with HIV infection, see U.S. 61/903,182 (“System And Method For The Detection and Treatment of Infection by a Microbial Agent Associated With HIV Infection”, expressly incorporated herein by reference). This amplicon corresponds to a sequence of human genomic origin (chromosome 1) but is carried by the bacterial co-factor present in red blood cells.

However, the 194 bp LTR amplicon from HIV 1, which is also an emitter of EMS, and was shown by the inventors and also in other distant laboratories (see, www.waterjournal.org/uploads/vol5/supplement/Montagnier.pdf, expressly incorporated herein by reference) to be transduced by its own EMS, did not induce cytopathic effect in HL60 cells, nor any DNA synthesis in these cells.

In addition, the 16S DNA amplicon of Sutterella (See, US 20120207726, WO/2013/139861, expressly incorporated herein by reference) which was shown by the inventors in earlier work to be present in the blood of autistic children, did not induce EMS nor any effects on HL60 cells.

Therefore, a method is now provided to transmit through recordable and digitizable electromagnetic signals, a DNA sequence in living cultivated cells, wherein the signal and/or the DNA sequence have a specific biological effect.

EMS have been detected in prepared samples from clinical specimens from patients suffering from certain chronic diseases. This would indicate that these EMS may play a role or be indicative of a process related to the persistence of the infectious agents and may contribute to their pathogenic effects. There is some evidence that DNA extracted from some tissues in certain chronic diseases has free radical modifications, and as discussed above, a number of prior researchers have associated free radical effects with EMS interactions.

Moreover, the successful transduction in living cells of the DNA specified by the EMS would indicate that such cells do possess the enzymatic capacity (DNA polymerase) to read the water nanostructures which represent the DNA sequence which is used to create the EMS. This property is therefore not a unique characteristic of the TAQ polymerase used in PCR, and may play a role in natural living organisms under physiological and pathogenic conditions.

TABLE 1

| | DNA | Cytopathic effect | | DNA in cells | | |
|---|---|---|---|---|---|---|
| File | length | HL60 | Lymphocytes | HL60 | Lymphocytes | EMS |
| 16S ribosomal DNA of B. Burgdorferi | 499 bp | ++ | — | Yes | No | + |
| 16S ribosomal DNA of the bacterial | 700 bp | + | | Yes | | + |
| cofactor of HIV | | | | | | |
| Human sequence pRick of the bacterial | 400 bp | ++ | | Yes | | + |
| cofactor of HIV (See, U.S. 20130196939) | | | | | | |
| Same human sequence of genomic DNA | 400 bp | − | | No | | |
| 16S ribosomal DNA of Sutterella | 260 bp | − | | No | | − |
| LTR HIVLai | 194 bp | − | | No | | |

Various modifications and variations of the described methods, procedures, techniques, and compositions as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the art, are intended to be within the scope of the following claims.

Each document, patent application or patent publication cited by or referred to in this disclosure is incorporated by reference in its entirety.

What is claimed is:

1. A method of producing cytotoxicity, comprising:
amplifying DNA from a pathogen using polymerase chain reaction technology;
purifying the amplified DNA;
serially diluting and mixing the purified DNA in water, to generate a dilute DNA sample in a container;
receiving an electromagnetic signal from the container, using a receiver configured to capture electromagnetic emissions from the container over a frequency range of at least 100 Hz to 10,000 Hz;
optionally recording the received electromagnetic signal;
amplifying the received or optionally recorded electromagnetic signal; and
emitting the amplified electromagnetic signal in proximity to living cells.

2. The method according to claim 1, wherein the purified DNA is constituted as 2 ng/ml in water, and serially diluted over a range within 10⁻² to 10⁻¹⁵.

3. The method according to claim 1, wherein the received electromagnetic signal is digitally recorded for about 6 seconds over a bandwidth of at least 400 Hz to 4 kHz.

4. The method according to claim 1, wherein the pathogen is of a genus selective from the group consisting of Borrelia and Ricketsiales.

5. The method according to claim 1, wherein serial dilutions of the purified DNA are analyzed for significant electromagnetic emissions by comparison with control samples that do not have DNA, and an amplitude of emissions within a band of 1500-2000 Hz from the diluted purified DNA sample is quantitatively compared with control sample.

6. The method according to claim 1, wherein the pathogen comprises Borrellia burgdorferi and the living cells comprise transformed neoplastic cells, wherein the emission of the amplified electromagnetic signal in proximity to the transformed neoplastic cells is cytoxic to the transformed neoplastic cells.

7. The method according to claim 1, wherein said emitting the amplified electromagnetic signal comprises transducing the amplified signal with a copper coil having 3 layers of 420 spirals of copper wire over a bobbin length of 80 mm, an internal diameter of 50 mm, and a resistance of about 6 Ohms, and said amplifying comprises amplifying over a pass band from 10 Hz to 20 kHz, with a variable output power of up to 140 W RMS.

8. The method according to claim 1, wherein the living cells are exposed to the amplified electromagnetic signal having a field strength of about 5 microTesla for at least 3 days.

9. The method according to claim 8, further comprising using polymerase chain reaction technology to amplify DNA from the exposed living cells with primers adapted to amplify the DNA from the pathogen.

10. The method according to claim 1, wherein the amplifying of DNA from the pathogen using polymerase chain reaction technology comprises employing primers specific for a 16S gene of a prokaryotic pathogen.

11. The method according to claim 1, wherein signals from DNA of at least two different pathogens are received, and separately used as a source of the amplified electromagnetic signal in proximity to the living cells.

12. The method according to claim 1, wherein the amplified DNA has a length of at least 100 bp.

13. The method according to claim 2, wherein the pathogen is a prokaryote, and the amplified DNA from the pathogen corresponds to human DNA.

14. An system for inducing cytotoxicity, comprising:
a receiver configured to receive an electromagnetic signal from a container, using a receiver configured to capture electromagnetic emissions from the container over a frequency range of at least 100 Hz to 10,000 Hz;
an amplifier configured to amplify the received electromagnetic signal; and
an emitter configured to emit the amplified electromagnetic signal in proximity to living cells.

15. The system according to claim 14, further comprising a recorder configured to record the received electromagnetic signal for about 6 seconds over a bandwidth of at least 400 Hz to 4 kHz.

16. The system according to claim 14, further comprising an analyzer configured to analyze an amplitude of the electromagnetic signal received from the container.

17. The system according to claim 16, the analyzer is configured to determine whether the electromagnetic emissions exceed an amplitude threshold within a defined bandwidth.

18. The system according to claim 17, wherein the defined bandwidth comprises 1,500 Hz to 2,000 Hz.

19. The system according to claim 14, wherein the emitter comprises a copper coil having 3 layers of 420 spirals of copper wire over a bobbin length of 80 mm, an internal diameter of 50 mm, and a resistance of about 6 Ohms, and wherein the amplifier has a pass band from 10 Hz to 20 kHz, and a variable output power of up to 140 W RMS, such that the emitter is configured to emit the amplified electromagnetic signal having a field strength of about 5 microTesla.

20. A method of selectively inducing a cytotoxic response in neoplastic cells, comprising:
emitting an electromagnetic signal corresponding to electromagentic signal emissions of a pathogenic prokaryotic organism, having a region of magnetic field strength of at least 5 microTesla at frequencies below about 20 kHz; and
incubating the neoplastic cells in the region of magnetic field strength of at least 5 microTesla at frequencies below about 20 kHz for at least 3 days.


SEQUENCE LISTING (4 sequences)

SEQ ID NO: 1 - DNA, 20 bp, Borrelia burgdorferi
caatcyggactgagacctgc

SEQ ID NO: 2 - DNA, 20 bp, Borrelia burgdorferi
acgctgtaaacgatgcacac

SEQ ID NO: 3 - DNA, 24 bp, Unknown
  Ricketsiales
cctgagaagagatttaagaacaaa

SEQ ID NO: 4 - DNA, 22 bp, Unknown
  Ricketsiales
ccatatactgcttctanntgct